(19) United States
(12) Patent Application Publication
(10) Pub. No.: US 2022/0106631 A1
(43) Pub. Date: Apr. 7, 2022

(54) BARCODING IN DROPLETS FOR CELL-CELL INTERACTION AND SECRETED PROTEIN DETECTION AND ANALYSIS

(71) Applicant: Scribe Biosciences, San Francisco, CA (US)

(72) Inventors: Russell Cole, San Francisco, CA (US); Maithreyan Srinivasan, San Francisco, CA (US)

(21) Appl. No.: 17/493,312

(22) Filed: Oct. 4, 2021

Related U.S. Application Data
(60) Provisional application No. 63/087,671, filed on Oct. 5, 2020.

(51) Int. Cl. C12Q 1/6834; G01N 33/543; G01N 33/68

(52) U.S. Cl. CPC C12Q 1/6834; G01N 33/6845; G01N 33/54313

(57) ABSTRACT

The present invention provides compositions, systems, and methods for barcoding cells, beads, and secreted proteins in discrete entities (e.g. droplets) to allow sequencing data from such components that are separated during processing to be associated via the common barcodes. In some embodiments, the barcodes are tethered to the cell surface via a lipid, cholesterol, or antibody, or are attached to a surface molecule that moves from one cell to another via trogocytosis. In certain embodiments, such methods allow cell-cell interactions or secreted proteins in the discrete entity to be monitored.

The present application claims priority to U.S. Provisional Application Ser. No. 63/087,671, filed Oct. 5, 2020, which is herein incorporated by reference in its entirety.

### FIELD OF THE INVENTION

The present invention provides compositions, systems, and methods for barcoding cells, beads, and secreted proteins in discrete entities (e.g. droplets) to allow sequencing data from such components that are separated during processing to be associated via the common barcodes. In some embodiments, the barcodes are tethered to the cell surface via a lipid, cholesterol, or antibody, or are attached to a surface molecule that moves from one cell to another via trogocytosis. In certain embodiments, such methods allow cell-cell interactions or secreted proteins in the discrete entity to be monitored.

### BACKGROUND

The field of single cell analysis had enabled researchers to understand the diversity in phenotype and genotype among populations of cells. Flow cytometry has enabled individual cells to be profiled based on surface markers. Recent advances in microfluidics have enabled RNA, DNA, and epigenetic sequencing on a single cell level, leading to commercially available products and widespread adoption [1-3]. Microfluidics have also enabled single cells to be isolated and assayed for >20 secreted proteins [4]. Further advances in the field have led to the field of single cell multi-modal omics, the measurement of multiple different types of data from the same single cells [5].

Biology depends on complex interactions between different cell types, extracellular components, and exogenous factors. As tissues contain varied structural organization and cells themselves are heterogenous, the local conditions that a given cell experiences can vary significantly compared to the average properties of the tissue. Current methods for single cell analysis, by definition, are taken out of their original biological context and analyzed in isolation, losing direct linkage of cellular interactions to anything but the bulk properties of the original sample. A more granular understanding of cellular interactions requires methods to link together interacting cells and to carry forward this information into single cell analysis.

Efforts to parse out cell-cell interactions from bulk experiments have been largely bioinformatic in nature. One approach uses single-cell RNA-seq data to identify upregulated ligands and receptors on different cell types. This data is then used to create ligand-receptor interaction maps in the original bulk experiments [6]. This approach can be refined by deterministically flow sorting and co-sequencing pairs of adhered cells to better understand interacting transcriptional phenotypes [7]. Another approach utilizes spatial transcriptomics (ST) to sequence co-localized groups of 10-200 cells from a tissue slice. Single cell RNA-seq is performed on the sample in parallel and used to establish generalized transcriptomic profiles of each cell type. This information is used to re-analyze the ST data and parse out the composition of the ST sample and the behavior of individual cells therein [8]. Small assemblages of cells can also be randomly loaded into discrete microscale compartments, such as microwells or microfluidic droplets, after which compartment of interest can then be recovered and sequenced [9]. This is fundamentally a similar approach to ST, in that multiple cells are sequenced together and single cell data is modeled computationally.

Platforms capable of high throughput deterministic handling of single cells are currently limited.

### SUMMARY OF THE INVENTION

The present invention provides compositions, systems, and methods for barcoding cells, beads, and secreted proteins in discrete entities (e.g. droplets) to allow sequencing data from such components that are separated during processing to be associated via the common barcodes. In some embodiments, the barcodes are tethered to the cell surface via a lipid, cholesterol, or antibody, or are attached to a surface molecule that moves from one cell to another via trogocytosis. In certain embodiments, such methods allow cell-cell interactions or secreted proteins in the discrete entity to be monitored.

In some embodiments, provides herein are compositions comprising: a) a carrier fluid, b) a discrete entity in the carrier fluid, c) first and second cells in the discrete entity, and d) a plurality of lipid/cholesterol-modified oligonucleotide complexes (LCMOCs) integrated into the cell membrane the first cell, wherein each of the LCMOCs: i) comprise a nucleic acid sequence comprising the same barcode region and a lipid and/or cholesterol moiety linked to the nucleic acid sequence, and ii) are configured to randomly exchange into the carrier fluid and integrate into the cell membrane of the second cell.

In certain embodiments, provided herein are methods comprising: a) merging first and second discrete entities in a carrier fluid in a microfluidics device such that a combined discrete entity is formed, wherein the first discrete entity contains a first cell, and wherein a plurality of lipid-modified oligonucleotide complexes (LCMOCs) are integrated into the cell membrane of the first cell, wherein each of the LCMOCs comprise a nucleic acid sequence comprising the same barcode region a lipid or cholesterol moiety linked to the nucleic acid sequence, and wherein the second discrete entity contains the second cell, and no other cells are present in the second discrete entity, and wherein no LCMOC's are present in the second discrete entity; and b) incubating the combined discrete entity such that at least one of the plurality of LCMCOs exchanges into the carrier fluid from the first cell membrane and then integrates into the cell membrane of the second cell.

In certain embodiments, each of the LCMOCs comprises: i) a first lipid-conjugated oligonucleotide comprising a first lipid moiety, a first hybridization region, and a first primer region; ii) a second lipid-conjugated oligonucleotide comprising a second hybridization region and a second lipid moiety, wherein the second hybridization region is the reverse complement of the first hybridization region; and iii) the nucleic acid sequence comprising the barcode region further comprising: a second primer region, and a capture sequence, wherein the second primer region is the reverse complement of the first primer region.

In some embodiments, one of the first and second cells is a T-cell and the other is an antigen presenting cell. In certain embodiments, the methods further comprise: d) identifying the first and second sequence information as originating from the combined discrete entity based on matching the sequence of the barcode region. In other embodiments, the

In particular embodiments, provided herein are compositions comprising: a) a carrier fluid, b) a discrete entity in the carrier fluid, c) first and second cells in the discrete entity, and wherein the first cell comprises a plurality of barcoded surface molecules (BCSMs) that are able to transfer from the first cell to the second cell when the first and second cells specifically interact, and wherein each of the BCSMs: i) comprises a surface molecule, and ii) a nucleic acid sequence comprising a barcode region, wherein the nucleic acid sequence is tethered to the surface molecule. In certain embodiments, the first and second cells specifically interact by antigen-specific contact. In other embodiments, the first and second cells specifically interact via a T-cell receptor on one of the cells binding to a cognate peptide ligand (epitope) presented on a major histocompatibility complex (MHC) protein molecule on the other cell. In additional embodiments, the first cell is a T-Cell and the second cell is an antigen presenting cell (APC). In other embodiments, the surface molecule comprises a protein.

In some embodiments, provided herein are methods comprising: a) merging first and second discrete entities in a carrier fluid in a microfluidics device such that a combined discrete entity is formed, wherein the first discrete entity contains a first cell, and wherein the first cell comprises a plurality of barcoded surface molecules (BCSMs), and wherein each of the BCSMs: i) comprises a surface molecule, and ii) a nucleic acid sequence comprising a barcode region, wherein the nucleic acid sequence is tethered to the surface molecule, wherein the second discrete entity contains the second cell, and wherein i) no other cells are present in the second discrete entity, or ii) one or more additional cells are present in the second discrete entity, and wherein no BCSM's are present in the second discrete entity; and b) incubating the combined discrete entity such that at least one of the plurality of BCSMs transfer from the first cell to the second cell when the first and second cells specifically interact.

In certain embodiments, the first and second cells specifically interact by antigen-specific contact. In some embodiments, the first and second cells specifically interact via a T-cell receptor on one of the cells binding to a cognate peptide ligand (epitope) presented on a major histocompatibility complex (MEW) protein molecule on the other cell. In other embodiments, the first cell is a T-Cell and the second cell is an antigen presenting cell (APC). In other embodiments, the surface molecule comprises a protein.

In some embodiments, the methods further comprise: c) processing the combined discrete entity such that: i) first sequencing information is obtained from the first cell that is associated with the sequence of the barcode region, and ii) separately, second sequence information is obtained from the second cell that is associated with the sequence of the barcode region. In other embodiments, the methods further comprise: d) identifying the first and second sequence information as originating from the combined discrete entity based on matching the sequence of the barcode region.

In certain embodiments, provided herein are compositions or systems comprising: a) a carrier fluid, b) first and second discrete entities, or a combined discrete entity, in the carrier fluid, c) first and second cells in the first discrete entity or in the combined discrete entity, and d) a plurality of antibody-linked oligonucleotides (ALOs) in the second discrete entity, or in the combined discrete entity, wherein each ALO: i) comprise a nucleic acid sequence comprising the same barcode region, and ii) an antibody, or a binding region of the antibody, linked to the nucleic acid sequence, wherein the antibody and the binding region specifically bind to the surface of the first and second cells.

In some embodiments, provided herein are methods comprising: a) merging first and second discrete entities in a carrier fluid in a microfluidics device such that a combined discrete entity is formed, wherein the first discrete entity contains first and second cell, and wherein the second discrete entity contains a plurality of antibody-linked oligonucleotides (ALOs), wherein each ALO: i) comprises a nucleic acid sequence comprising the same barcode region, and ii) an antibody, or a binding region of the antibody, linked to the nucleic acid sequence; and b) incubating the combined discrete entity such that at least some of the ALOs specifically bind to the surface of the first cell, and at least some of the ALOs bind to the surface of the second cell.

In particular embodiments, the first cell is a T-Cell and the second cell is an antigen presenting cell (APC). In other embodiments, the methods further comprise: c) processing the combined discrete entity such that: i) first sequencing information is obtained from the first cell that is associated with the sequence of the barcode region, and ii) separately, second sequence information is obtained from the second cell that is associated with the sequence of the barcode region. In other embodiments, the methods further comprise: d) identifying the first and second sequence information as originating from the combined discrete entity based on matching the sequence of the barcode region.

In certain embodiments, the nucleic acid sequence further comprises: a first amplification handle and a first anchor. In other embodiments, the first anchor comprises a poly-A sequence. In further embodiments, the first and second cells, and the plurality of ALOs, are present in the combined discrete entity, and a portion of the ALOs are bound to the surface of the first cell, and a portion of the ALO's are bound to the surface of the second cell. In additional embodiments, the first and second cells are present in the first discrete entity, and the plurality of ALOs are present in the second discrete entity. In other embodiments, one of the first and second cells is a T-cell and the other is an antigen presenting cell.

In some embodiments, provided herein are compositions and systems comprising: a) a carrier fluid, b) first and second discrete entities, or a combined discrete entity, in the carrier fluid, c) first and second cells in the first discrete entity or in the combined discrete entity, wherein the first and second cells each comprise an oligonucleotide construct attached to the cell surface, wherein the oligonucleotide construct comprises a hybridizability region and an attachment moiety, and d) a plurality of hybridizing oligonucleotides in the second discrete entity, or in the combined discrete entity, wherein each hybridizing oligonucleotide: i) comprise a nucleic acid sequence comprising the same barcode region, and ii) a complementary nucleic acid sequence configured to hybridize to the hybridizability region of the oligonucleotide construct.

In certain embodiments, provided herein are methods comprising: a) merging first and second discrete entities in a carrier fluid in a microfluidics device such that a combined discrete entity is formed, wherein the first discrete entity contains first and second cells, and wherein the first and second cells each comprise an oligonucleotide construct attached to the cell surface, wherein the oligonucleotide construct comprises a hybridizing region and an attachment moiety, and wherein the second discrete entity contains a plurality of hybridizing oligonucleotides, wherein each hybridizing oligonucleotide: i) comprise a nucleic acid sequence comprising the same barcode region, and a complementary nucleic acid sequence configured to hybridize to the hybridizing region of the oligonucleotide construct; and b) incubating the combined discrete entity such that at least some of the plurality of hybridizing oligonucleotides hybridize to the oligonucleotide constructs to thereby label the first and second cells with barcode sequences.

In particular embodiments, the attachment moiety comprises an antibody or binding region of the antibody. In other embodiments, the attachment moiety comprises a lipid moiety or a cholesterol moiety. In further embodiments, one of the first and second cells is a T-cell and the other is an antigen presenting cell. In some embodiments, the methods further comprise: c) processing the combined discrete entity such that: i) first sequencing information is obtained from the first cell that is associated with the sequence of the barcode region, and ii) separately, second sequence information is obtained from the second cell that is associated with the sequence of the barcode region. In additional embodiments, the methods further comprise: d) identifying the first and second sequence information as originating from the combined discrete entity based on matching the sequence of the barcode region.

In some embodiments, provided herein are compositions comprising: a) a carrier fluid, b) a discrete entity in the carrier fluid, c) a cell and a secreted protein from the cell in the discrete entity, d) a capture bead configured to bind to the secreted protein in the discrete entity, and e) a plurality of antibody-linked oligonucleotides (ALOs) in the discrete entity, wherein each ALO: i) comprises a nucleic acid sequence comprising the same barcode region, and ii) an antibody, or a binding region of the antibody, linked to the nucleic acid sequence, wherein the antibody, and the binding region, specifically bind to the secreted protein.

In certain embodiments, provided herein are methods comprising: a) merging first, second, and third discrete entities in a carrier fluid in a microfluidics device such that a combined discrete entity is formed, wherein the first discrete entity contains a cell that secretes a protein of interest, wherein the second discrete entity contain a bead that binds to the protein of interest, and wherein the third discrete entity contains a plurality of antibody-linked oligonucleotides (ALOs), wherein each ALO: i) comprises a nucleic acid sequence comprising the same barcode region, and ii) an antibody, or a binding region of the antibody, linked to the nucleic acid sequence, wherein the antibody, or binding region of the antibody, binds the protein of interest; and b) incubating the combined discrete entity such that: i) the cell secretes the protein of interest, ii) the bead binds the protein of interest, and iii) an the binds to the protein of interest.

In certain embodiments, the cell and/or the capture bead are labeled with a moiety linked to a barcoded nucleic acid sequence, wherein the moiety is selected from: a lipid, cholesterol, a surface molecule, an antibody, or a binding region of the antibody. In other embodiments, the secreted protein is selected from the group consisting of: a cytokine, a hormone, an enzyme, a toxin, and an antimicrobial peptide. In additional embodiments, the cell is a T-Cell or an antigen presenting cell (APC). In other embodiments, the methods further comprise: c) processing the combined discrete entity such that: i) first sequencing information is obtained from the cell, and ii) separately, second sequence information is obtained from the barcode region. In other embodiments, the methods further comprise: d) identifying the second sequence, and therefore the secreted protein, as originating from the cell in the combined discrete entity.

In some embodiments, the cell and the capture bead are labeled with a moiety linked to a barcoded nucleic acid sequence, wherein the moiety is selected from: a lipid, cholesterol, a surface molecule, an antibody, or a binding region of the antibody. In other embodiments, the methods further comprise: c) processing the combined discrete entity such that: i) first sequencing information is obtained from the cell that is associated with the sequence of the barcode region, and ii) separately, second sequence information is obtained from the capture bead that is associated with the sequence of the barcode region. In certain embodiments, the methods further comprise: d) identifying the first and second sequence information as originating from the combined discrete entity based on matching the sequence of the barcode region.

In some embodiments, provided herein are compositions and systems comprising: a) a carrier fluid, b) first, second, and third discrete entities, or a combined discrete entity, in the carrier fluid, wherein the combined discrete entity is composed of the first discrete entity and either the second or third discrete entity, c) first and second cells in the first discrete entity or in the combined discrete entity, d) a plurality of first antibody-linked oligonucleotides (1st-ALOs) in the second discrete entity, or in the combined discrete entity, wherein each 1st-ALO: i) comprise a first nucleic acid sequence comprising a first barcode region, and ii) a first antibody, or a binding region of the first antibody, linked to the first nucleic acid sequence, wherein the first antibody and the binding region specifically bind to the surface of the first and second cells; and e) a plurality of second antibody-linked oligonucleotides (2nd-ALOs) in the third discrete entity, or in the combined discrete entity, wherein each 2nd-ALO: i) comprise a second nucleic acid sequence comprising a second barcode region different from the first barcode region, and ii) a second antibody, or a binding region of the second antibody, linked to the second nucleic acid sequence, wherein the second antibody and the binding region specifically bind to the surface of the first and second cells.

In certain embodiments, the first cell is able to be activated by the second cell to release a detectable signal. In other embodiments, the first discrete entity further comprises detection reagents to detect the detectable signal. In additional embodiments, the first and second cells in the combined discrete entity are labeled with the 1st-ALOs. In some embodiments, the first and second cells in the combined discrete entity are labeled with the 2nd-ALOs. In further embodiments, one of the first and second cells is a T-cell and the other is an antigen presenting cell.

In further embodiments, provided herein are methods comprising: a) detecting a negative or positive cell-interaction assay signal from a first discrete entity, wherein the first discrete entity comprises first and second cells and cell-interaction detection reagents, b) performing one of the following: i) merging the first discrete entity with a second discrete entity in a carrier fluid in a microfluidics device if the positive cell-interaction assay signal is detected to generate a first combined discrete entity, wherein second discrete entity comprises a plurality of first antibody-linked oligonucleotides (1st-ALOs), wherein each 1st-ALO i) comprise a first nucleic acid sequence comprising a first barcode region, and ii) a first antibody, or a binding region of the first antibody, linked to the first nucleic acid sequence, or ii) merging the first discrete entity with a third discrete entity in a carrier fluid in a microfluidics device if the negative cell-interaction assay signal is detected to generate a second combined discrete entity, wherein the third discrete entity comprises a plurality of second antibody-linked oligonucleotides (2nd-ALOs), wherein each 2nd-ALO i) comprise a second nucleic acid sequence comprising a second barcode region different from the first barcode region, and ii) a second antibody, or a binding region of the second antibody, linked to the second nucleic acid sequence, and c) incubating the first combined discrete entity such that at least some of the 1st-ALOs bind to the surface of the first and second cells, or incubating the second combined discrete entity such that at least some of the 2nd-ALOs bind to the surface of the first and second cells.

In additional embodiments, the first cell is a T-Cell and the second cell is an antigen presenting cell (APC). In some embodiments, the methods further comprise: c) processing the first or second combined discrete entity such that: i) first sequencing information is obtained from the first cell that is associated with the sequence of the first or second barcode region, and ii) separately, second sequence information is obtained from the second cell that is associated with the sequence of the first or second barcode region. In additional embodiments, the methods further comprise: d) identifying the first and second sequence information as originating from the combined discrete entity based on matching the sequence of the first or second barcode regions.

In some embodiments, the discrete entity (or discrete entities) is/are droplets. In certain embodiments, the droplets comprise an aqueous fluid which is immiscible in the carrier fluid. In further embodiments, the carrier fluid comprises oil. In particular embodiments, the carrier fluid is an aqueous fluid and the droplet comprises a fluid which is immiscible with the carrier fluid. In additional embodiments, the discrete entity has a diameter of from about 1 μm to 1000 μm; and/or wherein the discrete entity has a volume of from about 1 femtoliter to about 1000 nanoliters, or from 10 to 800 picoliters. In other embodiments, i) no other cells are present in the discrete entity or entities (e.g., first, second and third discrete entities, or combined discrete entity), or ii) additional cells are present in the discrete entity (e.g., first, second, and third discrete entities, or combined discrete entity).

### DESCRIPTION OF THE FIGURES

FIG. 1 shows an exemplary droplet with only two cells. A plurality of surface tethered barcode oligonucleotides (e.g., that comprise a lipid or cholesterol moiety and nucleic acid sequence with barcode) are present on the first cell. At least one of these barcode oligonucleotides exchanges into the media of the droplet (e.g., oil or aqueous phase) and becomes tethered to a second cell in the droplet.

FIG. 2 shows an exemplary droplet with only two cells. The first cell has a plurality of surface molecules (not visible) that are labeled with nucleic acid sequences comprising a barcode sequence. When the two cells specifically interact, one or more of the barcoded surface molecules transfers to the second cell (e.g. by trogocytosis), thereby labeling the second cell with a barcode. Later sequence analysis allows one to determine that these two cells specifically interacted as they have the same barcode sequence.

FIG. 3 shows an exemplary droplet like FIG. 2, except there are more than two cells.

FIG. 4 shows an exemplary first droplet with two cells, and an exemplary second droplet with a plurality of constructs composed of antibodies linked to barcoded oligonucleotides. FIG. 4 shows the combination of the first and second discrete entities forms a combined discrete entity where constructs can bind the surface of the first and second cells via antibody recognition.

FIG. 5 shows an exemplary first droplet with two cells comprising an oligonucleotide construct attached to the their surfaces, and an exemplary second droplet with a plurality of hybridizing constructs configured to hybridize to the oligonucleotide constructs once the first and second droplets are merged.

FIG. 6 shows a discrete entity with a cell secreting a protein of interest (IL2), a bead that binds the protein of interest, and a construct comprising a barcoded oligonucleotide attached to an antibody (or binding fragment thereof) that binds the protein of interest.

FIG. 7 shows a discrete entity with a cell secreting a protein of interest (where the cell is labeled with a barcoded oligonucleotide), a bead that binds the protein of interest (where the bead is labeled with a barcoded oligonucleotide), and a construct comprising a barcoded oligonucleotide attached to an antibody (or binding fragment thereof) that binds the protein of interest.

FIG. 8 shows an exemplary embodiment where two cells in a first discrete entity (e.g., droplet), along with detection reagents, is incubated, then a positive or negative assay signal (e.g., IFNy signal) is detected (see “Assay Readout”). If the assay signal is positive, the first discrete entity is merged with a second discrete entity that contains constructs to label the cell(s) with a first type of barcode (see top row). If the assay signal is negative, the first discrete entity is merged with a third discrete entity that contains constructs to label the cell(s) with a second type of barcode that is different from the first type of barcode (see bottom row). When single cells are sequenced, sequencing the surface-tethered barcode provides information about the detected assay result.

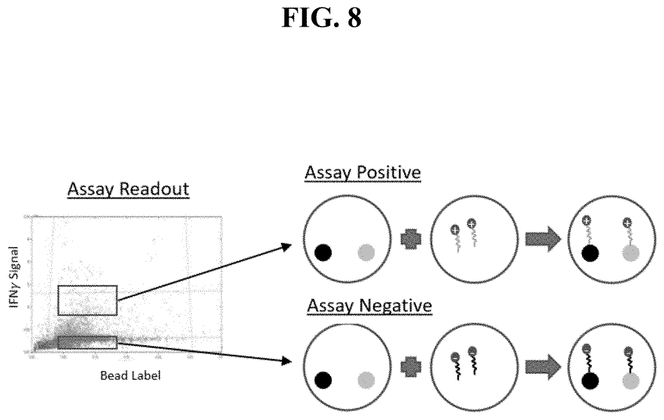

FIG. 9 provides a block schematic diagram of an example microfluidic device having an inlet channel, a sorting channel, a sorting element, first and second outlet channels, a trapping element, a discrete entity merger region, and upstream and a downstream regions.

FIG. 10 provides an image of a microfluidic device having a spacer fluid channel, a bias fluid channel, a laminating oil inlet channel, a concentric sorter channel, a flow divider, and a recess according to embodiments of the present disclosure.

FIG. 11 provides images of a microfluidic device having a concentric sorter channel, a recess, and an approximately triangular downstream region according to embodiments of the present disclosure.

FIG. 12, panels i-iv, show a zoomed-out view of an integrated droplet sorter-combiner. A droplet with a desired fluorescent signature is detected as it enters the droplet sorting region (i), the sorting electrode is actuated to redirect the drop towards the assembly lane (ii), and the sorted droplet merges with the droplet-in-assembly at the DEP trap (iii). Following assembly, the DEP trap is turned off to release the droplet (iv). Panels v-viii show a close-up of the merging process. 4 droplets are sorted by their fluorescent signature (pseudocolored) and directed to the DEP trap for merging (v). As the droplets encounter the actuated trap, they are sequentially merged into the assembled droplet (vi-vii). The electrode is then temporarily turned off so the assembled droplet may be released and recovered downstream (viii).

FIG. 13 provides a schematic flow diagram of a method of selectively combining discrete entities using a microfluidic device according to embodiments of the present disclosure.

FIG. 14 provides a schematic showing example configurations for trapping a discrete entity. Panel i) shows a bipolar electrode pair embedded in the same side wall of a channel. Panel ii) shows a bipolar electrode pair embedded on opposite sides of channel. Panel iii) shows bipolar electrode pair embedded in the floor or ceiling of a channel.

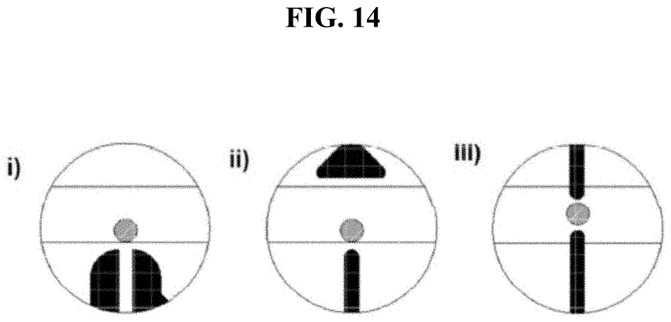

FIG. 15 provides a schematic showing example configurations for directing discrete entities to a discrete entity merger region. Panel i) shows application of a lamination flow to confine the laminar flow containing the droplet to the side wall of the channel. Panel ii) shows a partial height flow divider that allows fluid, but not droplets to enter the center portion of the channel. Panel iii) shows a configuration where a groove of similar height to the droplet dimensions is patterned near the side wall of a channel, while the rest of the channel is constructed with a reduced height to exclude droplets. Panel iv) shows a porous flow divider that allows fluid, but not droplets to enter the center portion of the channel. Panel v) shows a partial height flow dividers that direct droplets to a trap at the center of the microfluidic channel.

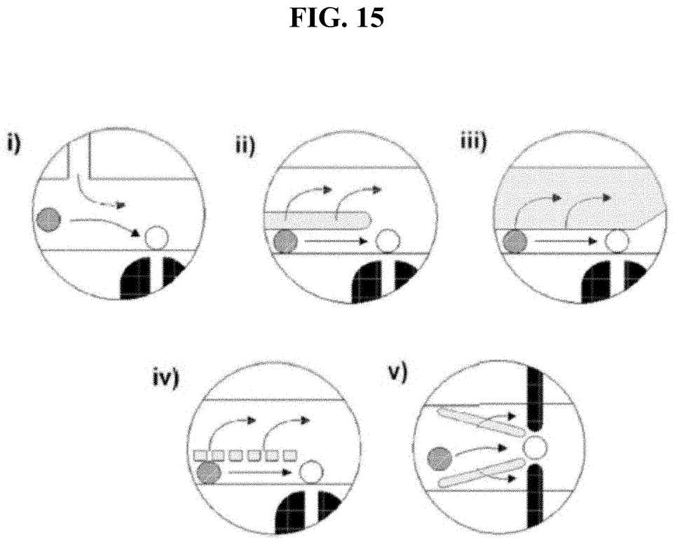

FIG. 16 provides a schematic showing an example embodiment wherein trapping is facilitated by a mechanical valve. Panel i) shows an initial stage where the discrete entities are trapped by the valve. Panel ii) shows a second stage wherein the discrete entities have been combined, e.g. due to electrical, chemical, or other means. Panel iii) shows a third stage where the combined discrete entity is released by opening the valve and carried downstream.

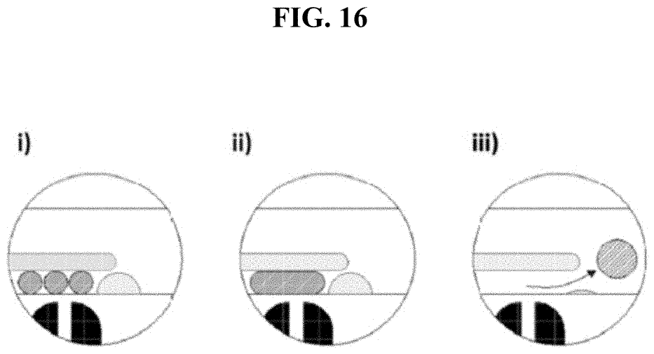

FIG. 17 provides a schematic showing example embodiments with different channel geometries in proximity to an electromagnetic trapping element. Panel i) shows a discrete entity merger region upstream of a bend in the channel wall. Panel ii) shows a discrete entity merger region in a lateral facet in the channel wall. Panel iii) shows a discrete entity being trapped in a region that is vertically taller than the main channel.

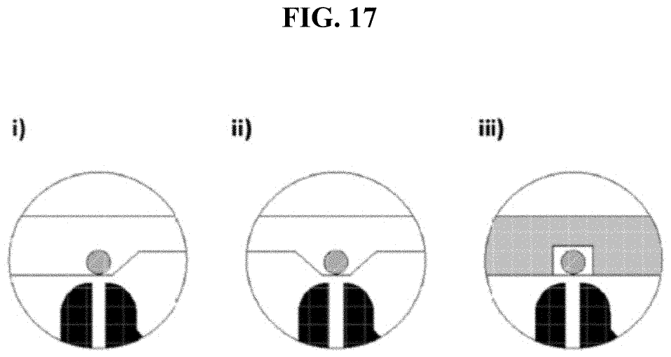

FIG. 18 shows a schematic representation of a lipid-modified oligonucleotide complex (“LMOC,” see WO2020010366) comprising an anchor lipid operably linked to the 5′ end of a first DNA oligonucleotide (i.e., an “anchor” lipid-modified oligonucleotide) comprising at a hybridization sequence and a primer region; a co-anchor lipid operably linked to the 3′ end of a second DNA oligonucleotide (i.e., a co-anchor lipid-modified oligonucleotide) comprising a hybridization sequence that is the reverse complement of the hybridization sequence of the first DNA oligonucleotide; and a third DNA oligonucleotide (i.e., a “barcode” oligonucleotide) comprising a primer region that is the reverse complement of the primer region of the first DNA oligonucleotide, a barcode region, and a capture sequence.

FIG. 19 shows an exemplary first cell that has a plurality of surface molecules (shown as stars) that are labeled with nucleic acid sequences comprising a barcode sequence. When the first cell specifically interacts with the second cell (e.g., via antigen recognition as shown), one or more of the barcoded surface molecules transfers to the second cell (e.g. by trogocytosis), thereby labeling the second cell with a barcode. Later sequence analysis allows one to determine that these two cells specifically interacted as they have the same barcode sequence.

### DETAILED DESCRIPTION

The present invention provides compositions, systems, and methods for barcoding cells, beads, and secreted proteins in discrete entities (e.g. droplets) to allow sequencing data from such components that are separated during processing to be associated via the common barcodes. In some embodiments, the barcodes are tethered to the cell surface via a lipid, cholesterol, or antibody, or are attached to a surface molecule that moves from one cell to another via trogocytosis. In certain embodiments, such methods allow cell-cell interactions or secreted proteins in the discrete entity to be monitored.

### I. Barcoding for Cell-Cell Interaction and Cell Protein Analysis

In certain embodiments, the present disclosure allows cell-cell interactions assays that are performed in small volumes, such as droplets or microwells to be labeled with common oligonucleotide barcodes. By labeling the cells contained in each microvolume with a unique barcode or combinations of barcodes, cells may be recovered in mass and run through existing single-cell sequencing workflows without loss of association between cells that are co-encapsulated. The re-association can be done bioinformatically through the recognition of common co-encapsulation barcodes on groups of single cells within the sequenced single-cell population.

In certain embodiments, the MOD microfluidic devices (described below in part II) are employed for the various barcoding techniques described herein. For example, droplet-based cell-cell interaction experiments using MOD (or similar device) can be sorted based on a positive assay signal, and a droplet containing a known barcode can be added to convey information about the assay result through sequencing. In certain embodiments, the identity of co-encapsulated cells are directly identified from single cell sequencing information.

A. First Exemplary Barcode Embodiments

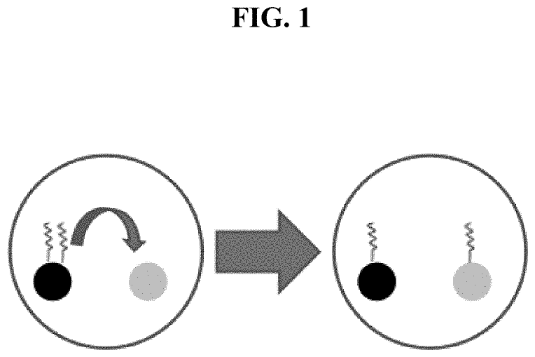

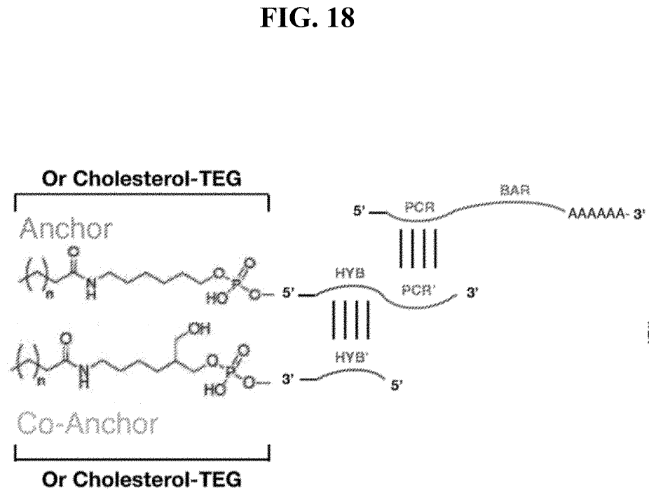

In certain embodiments, provided herein are compositions and methods for labeling a first cell with a plurality of surface tethered barcode oligonucleotides that, when in a discrete entity (e.g. droplet), some of which are exchanged into the media (e.g., oil or aqueous phase) in the discrete entity and become tethered to a second cell in the discrete entity (see FIG. 1). In some embodiments, the barcodes are part of a plurality of lipid or cholesterol-modified oligonucleotide complexes (LCMOCs) (e.g., composed of three oligonucleotides as shown in FIG. 18; see WO2020010366, herein incorporated by reference). In particular embodiments, only two cells are present in a discrete entity, and one or both cells are pre-barcoded with a unique oligo or combination of oligos prior to co-encapsulation in droplets. During incubation surface-tethered barcodes exchange into the droplet media and are pulled down onto the other cell. In some embodiments, such methods are carried out using a microfluidic device, such as the MOD devices described below. After breaking the emulsions, cells that are co-encapsulated maintain unique barcode signatures that can be used to associate previously co-encapsulated cells when they are sequenced individually.

B. Second and Third Exemplary Barcode Embodiments

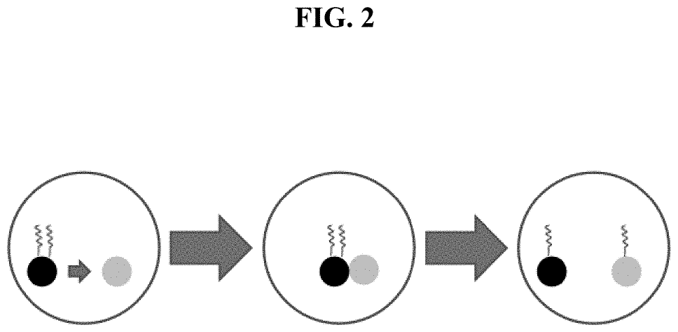

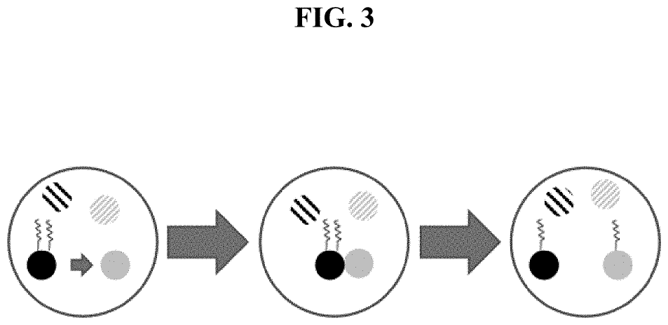

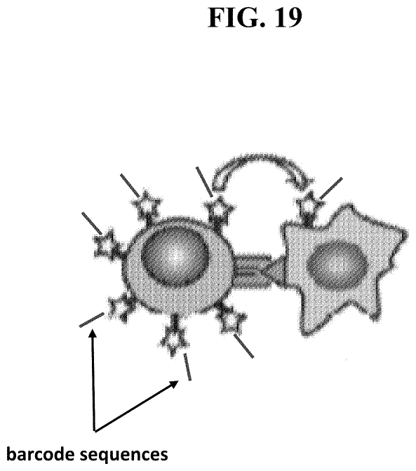

In certain embodiments, surface molecules that are transferred between cells when they interact (e.g., by trogocytosis) are labeled with unique barcode sequences. As shown in FIG. 2 and FIG. 19, when two cells (e.g., only two cells) are encapsulated in a discrete entity and interact, at least some of barcoded surface molecules are transferred from one cell to the other. Similarly, as shown in FIG. 3, there can be additional cells besides the first and second cells present in the discrete entity. One or both cells can be labelled in this manner prior to the interaction of the cells. Transfer of the barcodes allows one to confirm the cells interact and then trace such cells via later analysis. In some embodiments, such methods are carried out using a microfluidic device, such as the MOD devices described below. After breaking the emulsions, cells that are co-encapsulated maintain unique barcode signatures that can be used to associate previously co-encapsulated cells when they are sequenced individually. Certain trogocytosis methods are described in: Kula, et al., (2019), Cell, 178(4), 1016-1028.e13; Li, et al., (2019), Nature Methods, 16(2), 183-190; and WO2019023269; all of which are herein incorporated by reference in their entireties.

C. Fourth Exemplary Barcode Embodiments

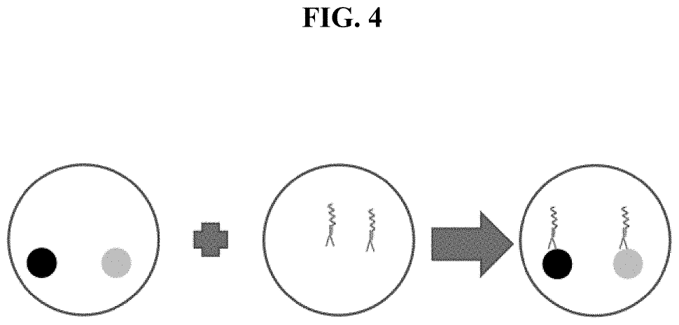

In certain embodiments, a first droplet with two cells is combined with a second droplet containing a plurality of constructs composed of antibodies (or other binding moiety, such as an aptamer) linked to barcoded oligonucleotides (e.g., as shown in FIG. 4). The combination of the first and second discrete entities (e.g., in a microfluidics device) forms a combined discrete entity where constructs bind the surface of the first and second cells via antibody recognition. Tagging the first and second cells with the same barcodes allows one to trace such cells via later analysis. In some embodiments, such methods are carried out using a microfluidic device, such as the MOD devices described below. After breaking the emulsions, cells that are co-encapsulated maintain unique barcode signatures that can be used to associate previously co-encapsulated cells when they are sequenced individually. References that described constructs with antibodies linked to barcoded nucleic acid sequences include: U.S. Pat. Pub. 201810251825, and Stoekius et al., Genome Biology (2018) 19:224, both of which are herein incorporated by reference, and particularly for the constructs described above.

D. Fifth Exemplary Barcode Embodiments

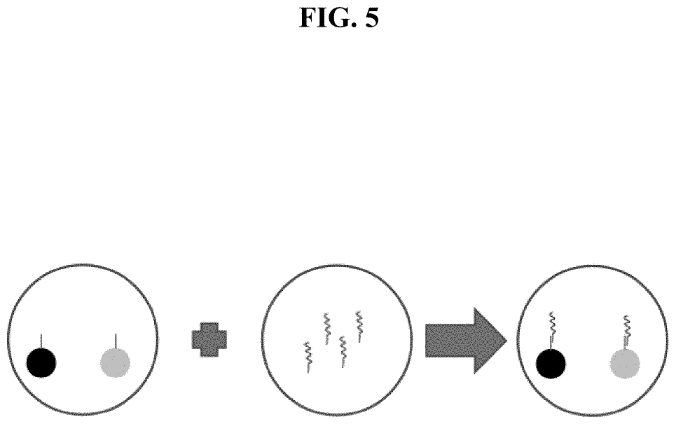

In certain embodiments, cells are pretreated with a construct with an attachment moiety to anchor a short, common oligo sequence to the surface of the cell (e.g., as shown in FIG. 5). The attachment moiety may be LMO anchors & co-anchors, cholesterol, antibodies or binding fragments thereof, or any other useful way to link oligos to cell surfaces. After co-incubation and before sequencing sample prep, the cell droplet is fused with a droplet containing oligos with a unique barcode signature and with a complimentary region to the nucleic acid sequence tethered to the cell surfaces (see FIG. 5). In some embodiments, such methods are carried out using a microfluidic device, such as the MOD devices described below. The fused droplets are incubated to allow hybridization, then broken and cells sequenced individually.

E. Sixth and Seventh Exemplary Barcode Embodiments

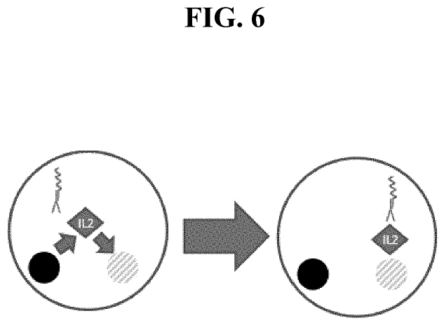

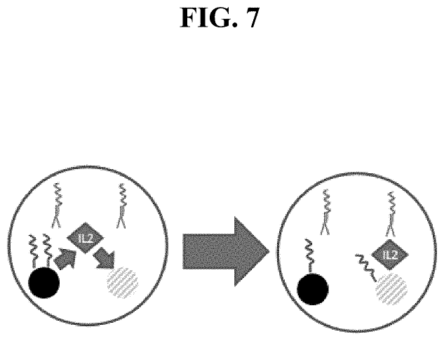

In certain embodiments, combined into a discrete entity are a cell secreting a protein of interest (e.g., a cytokine), a bead that binds the protein of interest, and a construct comprising a barcoded oligonucleotide attached to an antibody (or binding fragment thereof) that binds the protein of interest (e.g., as shown in FIG. 6). In some embodiments, such methods are carried out using a microfluidic device, such as the MOD devices described below. This discrete entity can be processed by breaking the emulsion, washing the cells and beads, then the content processed and sequenced. The secreted protein (e.g., cytokine) can be read out by sequencing individual beads. In some embodiments, the cell and/or the bead are co-barcoded by any of the methods described herein (see, FIG. 7). This allows individual cells to be linked to their secreted protein profiles.

F. Eighth Exemplary Barcode Embodiments

In certain embodiments, one or more cells in a first discrete entity (e.g., droplet), along with detection reagents, is incubated, then a positive or negative assay signal is detected. If the assay signal is positive, the first discrete entity is merged with a second discrete entity that contains constructs to label the cell(s) with a first type of barcode. If the assay signal is negative, the first discrete entity is merged with a third discrete entity that contains constructs to label the cell(s) with a second type of barcode that is different from the first type of barcode. When single cells are sequenced, sequencing the surface-tethered barcode provides information about the detected assay result. In some embodiments, such methods are carried out using a microfluidic device, such as the MOD devices described below.

### II. Exemplary Microfluidic Devices, Discrete Entities, and Biochemical Assays

In certain embodiments, the barcoding methods and compositions herein (e.g., eight exemplary barcode embodiments above) are employed with a microfluidic device that generates droplets (e.g., allowing single cell or limited number of cells to get interrogated). In some embodiments, the microfluidic device comprises a microenvironment on Demand (MOD) device, described in U.S. Provisional application Ser. No. 62/847,791, which is incorporated by reference herein. In general, the MOD platform is composed of an combination of: 1) a deterministic single-cell droplet sorter and droplet-assembler that can selectively assemble cells and reagents and 2) cell-based assays adapted to single-cell combinations in droplets.

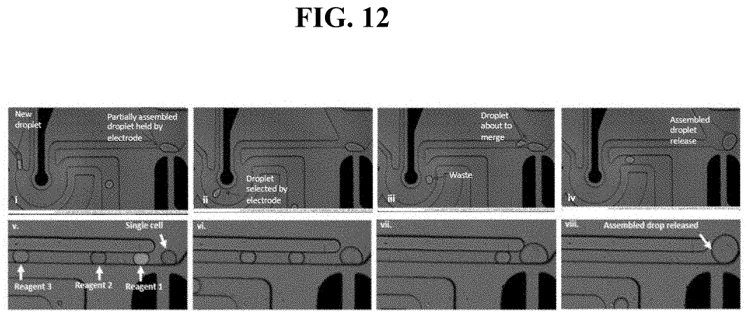

MOD performs a cyclic buildup and release of designer droplets through the merging of select droplets on a defined dielectrophoretic trapping position inside the microfluidic device (FIG. 12). This approach is advantageous because it is less prone to contamination, higher throughput, and requires fewer moving parts than other devices. The flexible nature of the MOD platform makes it a well-suited technology to perform integrated and functional cell-cell, cell-ECM interaction analysis and link any perturbations to select expressed gene sequences or transcriptome profiles at a single cell level. Essentially, MOD allows for precise, flexible, scalable liquid handling that can build a large number of predetermined reaction conditions.

MOD not only allows for the sorting and combination of particulates (cells, beads, hydrogels, etc.), but also sort and assemble diverse droplet contents (e.g., antibody solutions, cell stains, oligonucleotides etc.). Furthermore, droplet experiments constructed with MOD are compartmentalized and miniaturized (e.g., ˜100 pL) providing contained reactions in concentrated volumes. These two aspects of MOD, reagent selection and reaction miniaturization, provide a powerful approach to phenotypically screen large numbers of single cells. In some embodiments, the MOD platform couples cell-based assay results to sequencing readouts. Such coupling is used to obtain matched cell-cell sequence informatics.

Exemplary advantages of the MOD devices and systems include the following. First, querying larger numbers of cells allows access to a significant subset of the cell-cell interaction space (e.g., TCR-antigen interactions). As this space is more comprehensively mapped out, this information can, in some embodiments, be used to inform more generalized treatments. Second, the ability to build cell interaction experiments with single cell resolution enables more reproducible stimulation of cell-cell interactions than the uncontrolled local conditions in bulk experiments.

Discrete entities as used or generated in connection with the subject methods, devices, and/or systems may be sphere shaped or they may have any other suitable shape, e.g., an ovular or oblong shape. Discrete entities may be droplets. Discrete entities as described herein may include a liquid phase and/or a solid phase material. In some embodiments, discrete entities according to the present disclosure include a gel material. In some embodiments, the subject discrete entities have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, discrete entities as described herein have a dimension, e.g., diameter, of or about 1.0 μm to 5 μm, 5 μm to 10 μm, 10 μm to 100 μm, 100 μm to 500 μm, 500 μm to 750 μm, or 750 μm to 1000 μm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. In addition, discrete entities as described herein may have a size and/or shape such that they may be produced in, on, or by a microfluidic device and/or flowed from or applied by a microfluidic device.

In some embodiments, the discrete entities as described herein are droplets. The terms “drop,” “droplet,” and “microdroplet” are used interchangeably herein, to refer to small, generally spherically structures, containing at least a first fluid phase, such as an aqueous phase (e.g., water), bounded by a second fluid phase (e.g., oil) which is immiscible with the first fluid phase. In some embodiments, droplets according to the present disclosure may contain a first fluid phase (e.g., oil) bounded by a second immiscible fluid phase (e.g., an aqueous phase fluid, such as water). In some embodiments, the second fluid phase is an immiscible phase carrier fluid. Thus, droplets according to the present disclosure may be provided as aqueous-in-oil emulsions or oil in aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., barcoded sequences), enzymes, reagents, and a variety of other components. The term droplet may be used to refer to a droplet produced in, on, or by a microfluidic device and/or flowed from or applied by a microfluidic device.

As used herein, the term “dielectrophoretic force” refers to the force exerted on an uncharged particle caused by of the polarization of the particle by and interaction with a nonuniform electric field. A dielectrophoretic force can be directed towards (i.e. “attractive dielectrophoretic force”), away from (i.e. “repulsive dielectrophoretic force,”) or in any direction relative to the source of the electric field. Before being contacted by the electric field, the particle can be positively charged, negatively charged, or neutral.

As used herein, the term “electrophoretic force” refers to the force exerted on a charged particle caused by interaction with an electric field. An electrophoretic force can be directed towards (i.e. “attractive electrophoretic force”,) away from (i.e. “repulsive electrophoretic force,”) or in any direction relative to the source of the electric field. Before being contacted by the electric field, the particle can be positively charged, negatively charged, or neutral.

As used herein, the term “carrier fluid” refers to a fluid configured or selected to contain one or more discrete entities (e.g., droplets) as described herein. A carrier fluid may include one or more substances and may have one or more properties (e.g., viscosity), which allow it to be flowed through a microfluidic device or a portion thereof. In some embodiments, carrier fluids include, for example: oil or water, and may be in a liquid or gas phase.

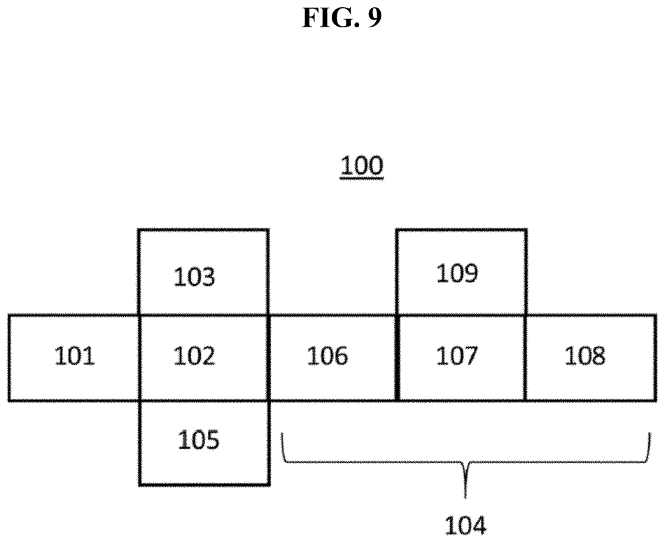

The present disclosure provides methods of selectively combining discrete entities (e.g., comprising barcoded nucleic acids sequences and cell(s)) using the MOD platform. FIG. 9 presents a non-limiting, simplified, schematic representation of one type of device and method according to the present disclosure. The microfluidic device of FIG. 9 is labeled as microfluidic device 100. FIG. 9 shows a representation of an inlet channel 101, wherein a discrete entity that is insoluble and/or immiscible in a carrier fluid a carrier fluid can be flowed through the inlet channel 101 to a sorter channel 102 that is in direct fluid communication with inlet channel 101. Next, the discrete entity can be sorted into a first outlet channel 104 or a second outlet channel 105, which are both in direct fluid communication with the sorter channel, by sorting element 103. Sorting element 103 can be, in some cases, an electrode, such as an electrode that is configured to exert a dielectrophoretic force on the discrete entity. Sorting element 103 in FIG. 9 is configured to sort a discrete entity in sorting channel 102 to first outlet channel 104 or second outlet channel 105. In some cases, if the discrete entity is sorted to second outlet channel 105, the discrete entity is sorted to a waste container or is recycled back to inlet channel 101. FIG. 9 shows an embodiment wherein first outlet channel 104 includes an upstream region 106, a discrete entity merger region 107, and a downstream region 108. In some cases, the discrete entity merger region comprises a change in a dimension of the first outlet channel, such as where the discrete entity merger region 107 has a larger cross-sectional area than the upstream region 106.

In addition, the FIG. 9 device includes trapping element 109. In some cases, trapping element 109 includes a trapping electrode, and the trapping electrode is configured to exert a force (e.g. a dielectrophoretic force), that traps the discrete entity in the discrete entity merger region 107. Furthermore, the discrete entity merger region 107 and the trapping element 109 are configured such that a force applied by the trapping electrode in the discrete entity merger region is sufficient to trap a plurality of discrete entities in the discrete entity merger region for a time sufficient for the plurality of discrete entities to combine to form a combined discrete entity. In some cases, a trapping electrode is configured to provide an electric field that affects the surface of the discrete entities such that the discrete entities can more easily merge (e.g. the discrete entities will spontaneously merge). In some cases, the affecting is destabilizing.

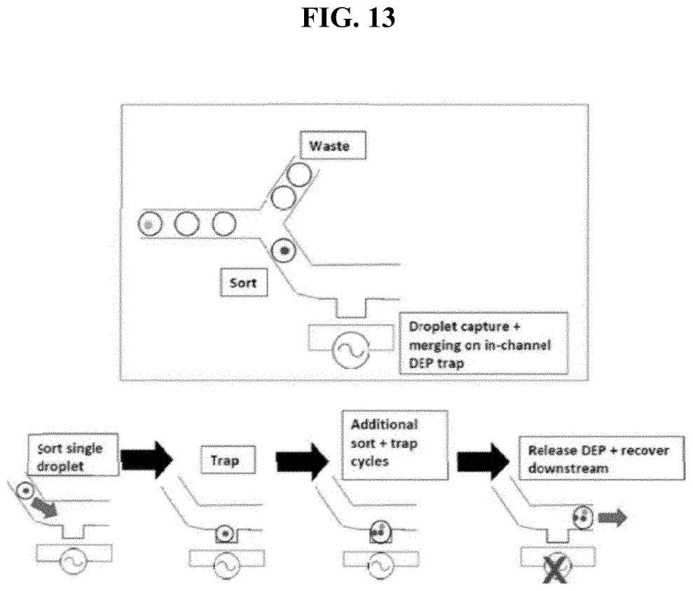

Methods of using the FIG. 9 device include flowing a plurality of discrete entities through inlet channel 101 to sorting channel 102, sorting with sorting element 103 the plurality of discrete entities into first outlet channel 104 or second outlet channel 105, trapping with trapping element 109 at least two discrete entities (e.g., one with a T-cell and one with an antigen presenting cell, presenting a neoantigen, both barcoded by methods herein) in discrete entity merger region 107 for a time sufficient for the at least two discrete entities to combine to form a combined discrete entity. FIG. 13 shows a schematic representation of an exemplary method wherein discrete entities containing cells are selectively combined.

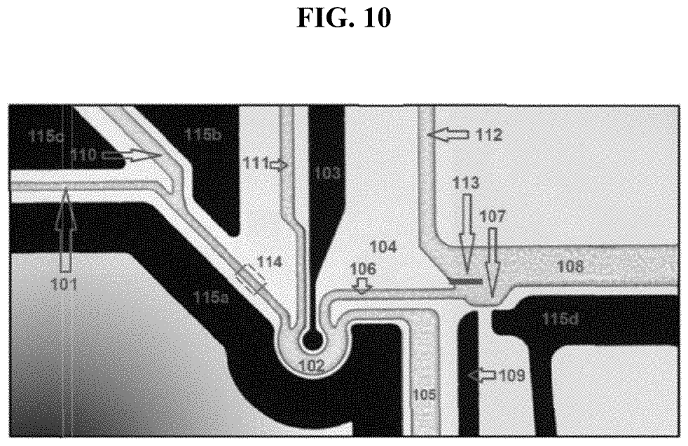

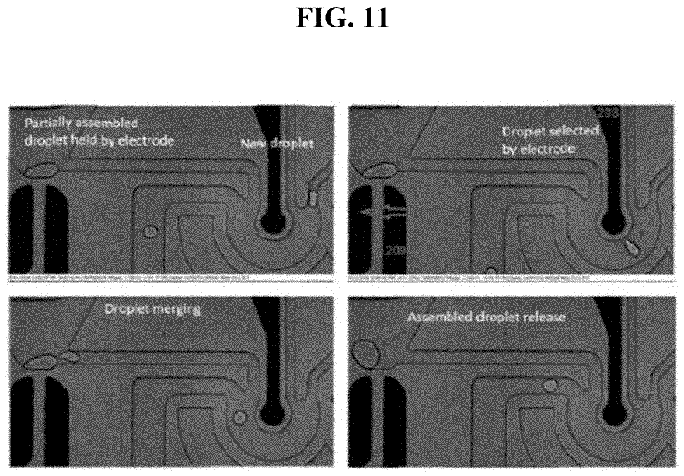

FIG. 10 presents an additional, non-limiting, simplified, schematic representation of one type of a device and method according to the present disclosure. In some cases, the discrete entity merger region includes a recess, such as shown as recess 107 in FIG. 10. In some cases, the discrete entity merger region includes a flow divider, such as shown as flow divider 113 in FIG. 10. In some cases, the device further includes a laminating oil inlet, such as shown as laminating oil inlet 112 in FIG. 10. In some cases, the trapping element includes two electrodes that have a significantly different shape from one another, such as shown as electrodes 109 in FIG. 10. In some cases, the trapping element includes two electrodes that produce a region of high electric field gradients that extends into the microfluidic channel. In some cases, the discrete entity merger region includes a change in the angle of flow between an adjacent upstream region and the discrete entity merger region, e.g. as shown in FIG. 11. In some cases, the device further includes a spacer fluid inlet. As an example, the device in FIG. 10 includes spacer fluid channel 110 in fluid communication with the inlet channel 101. The spacer fluid channel can be configured such that flowing spacer fluid through the spacer fluid channel causes spacer fluid to be located between two discrete entities flowing through the inlet channel, thereby maintaining or increasing the distance between the two discrete entities, thereby allowing each of the two discrete entities to be independently sorted or not sorted.

In some cases, the device further includes a bias fluid inlet. As an example, the device in FIG. 10 includes bias fluid channel 111 in fluid communication with sorter channel 102. The bias fluid channel can be configured such that flowing bias fluid through the bias fluid channel will cause a discrete entity to move closer to a second side wall of the sorter channel and farther away from a first side wall of the sorter channel. Thus, as an example, the spacer fluid inlet 111 would cause the discrete entity to move closer to the wall of the inlet channel that is closer to the bottom of the figure, and further away from the wall closer to the top of the figure. As such, one or more bias fluid channels can be configured such that a discrete entity will preferentially flow to a first outlet location or a second outlet location in the absence of a force from a sorting element. In some cases, the bias fluid inlet channel can be configured such that a discrete entity will preferentially flow to a second outlet channel in the absence of a dielectrophoretic force from a sorting electrode. As an example, the bias fluid inlet 111 in FIG. 10 causes a discrete entity to preferentially flow to second outlet channel 105 in the absence of a force exerted on the discrete entity by the sorting electrodes 103.

In some cases, the device includes a detector configured to detect a discrete entity in the input channel, wherein the microfluidic device is configured to sort a discrete entity in the sorting channel based on the detection by the detector. As an example, FIG. 10 shows an embodiment in which a discrete entity in detection region 114 of inlet channel 101 can be detected by a detector, after which sorting electrodes 103 can sort the discrete entity into the first outlet channel 104 or the second outlet channel 105. The FIG. 10 devices also includes shielding electrodes 115a, 115b, 115c, and 115d. As used herein, the term “shielding electrode” is used interchangeably with “moat electrode”. Each shielding electrode can be configured to perform one or more functions including: at least partially shielding discrete entities from undesired electromagnetic fields, assisting with the sorting of discrete entities, and assisting with the trapping of discrete entities.

As such, as used herein, shielding electrodes can also be referred to as sorting electrodes or trapping electrodes if such electrodes are configured to participate in the sorting or trapping of discrete entities. Hence, shielding electrode 115a can also be referred to as a sorting electrode if it is configured to form a bipolar electrode pair with sorting electrode 103 to facilitate the sorting of discrete entities. Similarly, shielding electrode 115d can also be referred to as a trapping electrode if it is configured to form a bipolar electrode pair with trapping electrode 109 to facilitate the trapping of discrete entities.

In some cases, a shielding electrode can generate an electromagnetic field such that discrete entities in the device is at least partially shielded from undesired electromagnetic fields. Such undesired electromagnetic fields can originate from outside the microfluidic device or from within the microfluidic device. In some cases, the undesired electromagnetic fields are those fields that are not generated by a sorting electrode or by a trapping electrode. By at least partially shielding discrete entities in the microfluidic device, the shielding electrodes can inhibit the unintended merging of discrete entities (i.e. merging of discrete entities outside the discrete entity merger region). In some cases, shielding electrodes 115a, 115b, and 115c can be used to at least partially shield discrete entities from electromagnetic fields that are not generated by the sorting electrode or the trapping electrode.

In some cases, shielding electrodes can assist with the sorting of discrete entities. As an example, shielding electrode 115a can interact with sorting electrode 103 in order to facilitate sorting, such as by forming a bipolar electrode pair with sorting electrode 103. In some cases, sorting electrode 103 can be the charged electrode (e.g. positively charged), and shielding electrode 115a can be a ground. Stated in another manner, shielding electrode 115a can be configured to influence the shape of the electromagnetic field generated by sorting electrode 103 in order to facilitate sorting.

In some cases, shielding electrodes can assist with the trapping of discrete entities. As an example, shielding electrode 115d can interact with trapping electrode 109 in order to facilitate trapping, such as by forming a bipolar electrode pair with trapping electrode 109. In some cases, sorting electrode 109 can be the charged electrode (e.g. positively charged), and shielding electrode 115d can be a ground. Stated in another manner, shielding electrode 115d can be configured to influence the shape of the electromagnetic field generated by trapping electrode 109 in order to facilitate sorting.

In some cases, one or more of the shielding electrodes are separate elements, such as when all the shielding electrodes are separate elements. In some cases, one or more of the shielding electrodes are directly electrically connected. In some cases, one or more of the shielding electrodes are different regions of a single electrode, such as part of a single piece of metal. In some cases, one or more of the shielding elements are attached to ground.

As shown in FIG. 10, in some cases, the device includes one or more shielding electrodes. In some cases, the device includes zero shielding electrodes, such as when the discrete entities are sorted using a single sorting electrode and the discrete entities are trapped using a single trapping electrode.

As such, discrete entities are sorted and selectively combined within a microfluidic device (i.e., without leaving the microfluidic device). Stated in another manner, the discrete entities are sorted and combined without leaving microfluidic sized channels and regions.

In addition, the present disclosure provides examples of specific elements and steps that can be used with the described devices, systems, and methods. As reviewed above, the trapping element and the sorting element can be electrodes that exert a dielectrophoretic force on the discrete entity. In some cases, the electrodes are microfluidic channels containing a conductive material (e.g. saltwater, liquid metal, molten solder, or a conductive ink to be annealed later). In some cases, the electrodes are patterned on the substrate of the microfluidic device (e.g. a patterned indium tin oxide (ITO) glass slide). In some cases, the trapping element includes two electrodes. In some cases, the trapping element is a selectively actuatable bipolar droplet trapping electrode. In some cases, the sorting element includes two electrodes. In some cases, the sorting element includes a selectively actuatable bipolar droplet sorting electrode.

In some cases, the sorting channel includes a partial height flow divider. In some cases, the sorting channel has a concentric or essentially concentric flow path and a portion of the sorting electrode is positioned at the center of the arc of the concentric or essentially concentric flow path.

In some embodiments, the discrete entity includes a particle (e.g. a cell, such as a T-cell or APC). In some embodiments, the discrete entity includes a chemical reagent (e.g. a lysing agent or a PCR reagent). In some embodiments, the discrete entity includes both a cell and a chemical reagent. In some embodiments, the discrete entity includes a fluorescently tagged T-cell or APC.

In some cases, the sorting is passive sorting. In some cases, the sorting is active sorting (i.e., the sorting element sorts a discrete entity into one of at least two locations based on a detected property of the discrete entity or a component within the discrete entity). In some cases, the detected property is an optical property and the device further includes an optical detector (e.g. an optical detector configured to detect an optical property of a discrete entity or a component within in the inlet channel). In some cases, the optical property is fluorescence and the device further includes a source of excitation light. In some cases, the sorting is based on the detected fluorescence of a fluorescent tag on a cell in the discrete entity.

In some cases, the discrete entity merger region can include structural elements that are configured to aid in the trapping and combination of discrete entities therein. In some cases, such structural elements are configured to aid in such trapping and combining by changing the speed or direction of the flow of fluid through an area of the discrete entity merger region.

The present disclosure also provides methods of using systems that include a microfluidic device, e.g. as described above, and one or more additional components, e.g. (a) a temperature control module operably connected to the microfluidic device; (b) a detector configured to detect a discrete entity in the input channel, wherein the microfluidic device is configured to sort a discrete entity in the sorting channel based on the detection by the detector; (c) an incubator operably connected to the microfluidic device or a discrete entity maker; (d) a sequencer operably connected to the microfluidic device; (e) a device configured to make a plurality of discrete entities, wherein the device is located within the microfluidic device or separately from the microfluidic device; and (f) one or more conveyors configured to convey a particle (e.g. a cell, or a discrete entity), wherein the discrete entity can contain a particle in some cases, between any combination of: the incubator, device configured to make a plurality of discrete entities, the microfluidic device, the sequencer.

In some cases, the methods include controlling the temperature of the microfluidic device using a temperature control module operably connected to the microfluidic device. In some cases, the methods include detecting a discrete entity in the input channel of the microfluidic device (e.g. detecting an optical property of the discrete entity or a component therein), and sorting the discrete entity based on the detecting. In some cases, the method includes incubating cells in an incubator that is operably connected to discrete entity maker or a microfluidic device. In some cases, the method includes making discrete entities with a discrete entity maker, wherein the discrete entity maker is located within the microfluidic device or separate from the microfluidic device. In some cases, the method includes moving a discrete entity between components of the system (e.g. with one or more conveyors).

The present disclosure also provides steps that can be performed after the release of a combined microfluidic droplet from a discrete entity merger region. In some cases, the method includes recovering a component (e.g. a barcoded cell, a chemical compound or a combination thereof), from the combined discrete entity. In cases where a combined discrete entity includes one or more cells, the one or more cells can be analyzed (e.g. genetic information therein, can be sequenced using a sequencer). The genetic information can include, e.g. barcodes, DNA and RNA. In some cases, the sequencing includes PCR. In some cases, the analysis of a discrete entity can include mass spectrometry. In some cases, the method includes printing the combined discrete entity onto a substrate, e.g. as described in US 2018/0056288, which is incorporated herein by reference for its disclosure of printing a discrete entity onto a substrate.

The present disclosure also provides a method of selectively performing reactions by selectively combining two or more discrete entities, as described above, wherein the reaction occurs between one or more components from each discrete entity. Such components can be one or more cells (e.g., barcoded cells), one or more products derived from a cell, one or more reagents, or a combination thereof. In some cases, the one or more products derived from a cell include cell lysate, DNA, RNA, or a combination thereof. As an example, FIG. 12 shows the combination of four discrete entities, wherein three of the discrete entities each contain a different reagent and the fourth discrete entity contains a single cell (e.g., APC or T-cell). As such, FIG. 12 shows that a microfluidic device as described herein can be used to selectively combine different discrete entities, resulting in the formation of a combined discrete entity (e.g., that contains the three reagents and the T-cell and/or APC).

As such, the method of selectively performing reactions can include the combination of two or more discrete entities (e.g. three or more and four or more), which allows a first cell (e.g., T-cell) and a second cell (e.g., APC) cell to be brought together and barcoded. In some cases, the number of discrete entities that contain at least one cell is zero discrete entities, one discrete entity, two discrete entities, or three or more discrete entities. In some cases, the number of cells in a discrete entity is one. In some cases, the method includes repeating the selective combination of discrete entities (e.g. performing the selective combination two or more times, three or more times, or four or more times, or 1000 or more times or a million or more times).

The present methods allow for the selective combination of two or more discrete entities without the need to accurately time the release or to accurate time the sorting of the two or more discrete entities. As such, in some cases, a first discrete entity is trapped in the discrete entity merger region before a second discrete entity to be combined therewith has entered the outlet channel after being sorted. In some cases, the second discrete entity has not entered the sorter channel, has not entered the inlet channel, or has not even been made when the first discrete entity is trapped in the discrete entity merger region.

The present methods allow for the sorting of discrete entities based on whether they contain a particular cell (e.g., barcoded cell), and allows the selective combination of only those discrete entities that contain the desired components. In some cases, the method involves creating 5 or more combined discrete entities per minute, including 10 or more, 25 or more, 50 or more, 75 or more, 100 or more, 150 or more, 200 or more, or 300 or more. In some cases, the method involves making 300 or more combined discrete entities per hour, including 1,500 or more, 3,000 or more, 4,500 or more, 6,000 or more, 9,000 or more, 12,000 or more, or 21,000 or more. In some cases, the sorting step is performed such that discrete entities are sorted at a rate of 0.01 Hz or more (e.g. 0.1 Hz or more, 1 Hz or more, 10 Hz or more, 100 Hz or more, 1 kHz or more, 10 kHz or more, or 30 kHz or more). In some cases, an electromagnetic sorter is used instead of a mechanical sorter (e.g. a valve, to allow for faster sorting rates). In some cases, the trapping and combining steps are performed such that a combined discrete entity is formed or released at a rate of 1 Hz or more, e.g. 10 Hz or more, 100 Hz or more, or 1,000 Hz or more.

In some cases, a discrete entity is flowed such that it reaches the discrete entity merger region between 0.1 ms to 1,000 ms after being sorted, such as between 1 ms and 100 ms, between 2 ms and 50 ms, and between 5 ms and 25 ms. In some cases, the first outlet channel is between 0.2 mm long and 5 mm long. In some cases, the first outlet channel has a dimension (e.g., width or height or diameter) of between 5 μm and 500 μm, such as between 10 μm and 100 μm. In some cases, the carrier fluid containing the discrete entities is flowed into the inlet channel at a rate of between 1 μl per hour and 10,000 μl per hour, such as between 10 μl per hour and 1,000 μl per hour, 25 μl per hour and 500 μl per hour, and between 50 μl per hour and 250 μl per hour. In some cases, the spacer fluid is injected at a rate of between 100 μl per hour and 20,000 μl per hour, such as 500 μl per hour and 5,000 μl per hour. In some cases, the bias fluid is injected at a rate of between 100 μl per hour and 20,000 μl per hour, such as 500 μl per hour and 5,000 μl per hour. In some cases, the fluid used to create cell-containing discrete entities has a concentration of between 1,000 cells per ml and 10,000,000 cells per ml, such as between 10,000 cells per ml and 1,000,000 cells per ml, and between 50,000 cells per ml and 200,000 cells per ml. In some cases, the discrete entities have a volume between 1 pl and 10,000 pl, such as between 10 pl and 1,000 pl, or between 50 pl and 500 pl.

In some cases, the one or more cells from a combined discrete entity are cultured for at least 30 minutes or more, such as 1 hour or more, 6 hours or more, 12 hours or more, 24 hours or more, 3 days or more, or 7 days or more. In some cases, the device can continuously operate by selectively combining discrete entities for 10 minutes or more, such as 30 minutes or more, 45 minutes or more, 90 minutes or more, or 180 minutes or more. In some cases, the device can make at least 100 combined discrete entities while continuously operating, such as 1,000 combined discrete entities or more, 10,000 combined discrete entities or more, or 100,000 combined discrete entities or more.

In some cases, the methods include making one or more discrete entities, such as with a discrete entity maker. In such cases, the discrete entity maker can be part of the microfluidic device or separate from the microfluidic device as otherwise described herein. If the discrete entity maker is separate from the microfluidic device, the discrete entity maker can be operably connected to the microfluidic device (e.g., such that discrete entities can flow from the maker to the microfluidic device), or the discrete entities can be moved to the microfluidic device without the discrete entity maker and microfluidic device being operably connected. The systems and devices can include one or more discrete entity makers configured to form discrete entities from a fluid stream. Suitable discrete entity makers include selectively activatable droplet makers and the methods may include forming one or more discrete entities via selective activation of the droplet maker. The methods may also include forming discrete entities using a droplet maker, wherein the discrete entities include one or more entities which differ in composition. In some cases, the discrete entity maker comprises a T-junction and the method includes T-junction drop-making. In some cases, making the discrete entities includes a step of emulsification. In some cases, the discrete entity maker is made, in part or in whole, of a polymer. In some cases, one or more surfaces of the discrete entity maker are coated with a fluorosilane (e.g. such a discrete entity maker can be used when fluorinated fluids are passed through the discrete entity maker).

In some cases when multiple types of discrete entities are made (e.g., discrete entities that contain different contents, such as one with a T-cell and one with an APC), the contents can affect the ability of the discrete entity maker to successfully make the discrete entities. As such, in some cases, different conditions for the discrete entity maker are used to make a first group of discrete entities with first contents than for making a second group of discrete entities with second contents.

Aspects of the disclosed methods may include making discrete entities using one or more cells from a biological sample. In such cases, each discrete entity may contain zero, one, or more than one cell. In some cases, such discrete entities can be made by incorporating the biological sample, cells from the biological sample, barcoded oligonucleotides, lysate from cells of the biological sample, or any other sample derived from the biological sample into a mixed emulsion. In some cases, the method further includes separating one or more components of the biological sample or otherwise processing the biological sample (e.g. via centrifugation, filtration, and the like), before making the discrete entities.

In some cases, after the making of the discrete entities but before introducing the discrete entities to an inlet channel of a microfluidic device as described herein, the discrete entities can be further modified (e.g. by adding a T-cell, APC, a reagent, a drug, a hydrogel, an extracellular matrix, a bead, a particle, a biological material, media, or a combination thereof). In some cases, the reagent is a primer, a probe, a lysing agent, a surfactant, a detergent, a barcode, or a fluorescent tag. In some cases, the bead is an RNA capture bead. In some cases, the bead is an immunoassay bead. In some cases, the barcode is an oligonucleotide. In some cases, different types of discrete entities are labeled with different types of barcodes, fluorescent tags, or a combination thereof.

Fluorescent tags can be used to image a discrete entity or combined discrete entity in the discrete entity merger region. Fluorescent tags can also be used to identify the particular type of discrete entities that were combined to create a given combined discrete entity. As such, the properties of the combined discrete entity or component thereof can be correlated with the contents that were used to make the original discrete entities. As an example, different types of T-cells can be labeled with different barcoded oligonucleotides and fluorescent labels and incorporated into discrete entities. After such T-cell-containing discrete entities are combined with other discrete entities (e.g. containing antigen presenting cells (APCs)), the outcome of the combined discrete entities can be observed (e.g., T-cell activation via cytokine release). As some of all of the original discrete entities can be labeled with fluorescent tags, the resulting combined discrete entity can have multiple fluorescent tags. In other cases, the combined discrete entity only has one fluorescent tag. Oligonucleotide barcodes can be used in a similar manner to that of fluorescent tags. Instead of detecting optical fluorescence, however, the oligonucleotide barcodes can be sequenced in order to identify the original discrete entities that formed the combined discrete entity.

Methods and devices which may be utilized in the encapsulating of a component from a biological sample are described in PCT Publication No. WO 2014/028378, the disclosure of which is incorporated by reference herein in its entirety and for all purposes. Encapsulation approaches of interest also include, but are not limited to, hydrodynamically-triggered drop formation and those described by Link, et al., Phys. Rev. Lett. 92, 054503 (2004), the disclosure of which is incorporated herein by reference. Other methods of encapsulating cells into droplets may also be applied. Where desired, the cells may be stained with one or more antibodies and/or probes prior to encapsulating them into drops.

One or more lysing agents may also be added to the discrete entities (e.g., droplets), containing a cell, under conditions in which the cell(s) may be caused to burst, thereby releasing their genomes. The lysing agents may be added after the cells are encapsulated into discrete entities. Any convenient lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities (e.g., droplets), may be heated to about 37-60° C. for about 20 min to lyse the cells and to allow the proteinase K to digest cellular proteins, after which they may be heated to about 95° C. for about 5-10 min to deactivate the proteinase K. In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient methods of effecting cell lysis may be employed in the methods described herein as appropriate.

One or more primers may be introduced into the discrete entities for each of the genes to be detected. Hence, in certain aspects, primers for all target genes may be present in the discrete entity at the same time, thereby providing a multiplexed assay. The discrete entities may be temperature-cycled so that discrete entities will undergo PCR. In certain embodiments, rolling circle amplification (RCA)-based proximity ligation is employed.

In some embodiments, a surfactant may be used to stabilize the discrete entities. In some cases, the discrete entities or the associated emulsion lack a surfactant. Accordingly, a discrete entity may involve a surfactant stabilized emulsion. Any convenient surfactant that allows for the desired reactions to be performed in the discrete entities, may be used. In other aspects, a discrete entity is not stabilized by surfactants or particles. The surfactant used depends on a number of factors such as the oil and aqueous phases (or other suitable immiscible phases (e.g., any suitable hydrophobic and hydrophilic phases)) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures. Other surfactants can also be envisioned, including ionic surfactants. Other additives can also be included in the oil to stabilize the discrete entities including polymers that increase discrete entity stability at temperatures above 35° C.

The discrete entities (e.g., microdroplets) described herein may be prepared as emulsions, such as an aqueous phase fluid dispersed in an immiscible phase carrier fluid (e.g., a fluorocarbon oil or a hydrocarbon oil) or vice versa. In some cases, the carrier fluid comprises a fluorinated compound. In some cases, the carrier fluid is an aqueous fluid. The nature of the microfluidic channel (or a coating thereon) (e.g., hydrophilic or hydrophobic), may be selected so as to be compatible with the type of emulsion being utilized at a particular point in a microfluidic workflow.

Emulsions may be generated using microfluidic devices. Microfluidic devices can form emulsions composed of droplets that are uniform in size. The microdroplet generation process may be accomplished by pumping two immiscible fluids, such as oil and water, into a junction. The junction shape, fluid properties (viscosity, interfacial tension, etc.), and flow rates influence the properties of the microdroplets generated but, for a relatively wide range of properties, microdroplets of controlled, uniform size can be generated using methods like T-junctions and flow focusing. To vary microdroplet size, the flow rates of the immiscible liquids may be varied since, for T-junction and flow focus methodologies over a certain range of properties, microdroplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating microdroplet of the desired size and rate.

In some cases, a cell in a discrete entity may be labeled (e.g., by a fluorescent label, a barcode, or a combination thereof). In practicing the subject methods, a number of reagents may be incorporated into and/or encapsulated by, the discrete entities in one or more steps (e.g., about 2, about 3, about 4, or about 5 or more steps). Such reagents may include, for example, amplification reagents, such as Polymerase Chain Reaction (PCR) reagents. The methods of adding reagents to the discrete entities may vary in a number of ways. Approaches of interest include, but are not limited to, those described by Ahn, et al., Appl. Phys. Lett. 88, 264105 (2006); Priest, et al., Appl. Phys. Lett. 89, 134101 (2006); Abate, et al., PNAS, Nov. 9, 2010 vol. 107 no. 45 19163-19166; and Song, et al. Anal. Chem., 2006, 78 (14), pp 4839-4849; the disclosures of which are incorporated herein by reference. For instance, a reagent may be added to a discrete entity by a method involving merging a discrete entity with a second discrete entity which contains the reagent(s) in a discrete entity merger region of a microfluidic device described herein.

One or more reagents may also, or instead, be added using techniques such as droplet coalescence, or picoinjection. In droplet coalescence, a target drop may be flowed alongside a microdroplet containing the reagent(s) to be added to the droplet. The two droplets may be flowed such that they are in contact with each other, but not touching other microdroplets. These drops may then be passed through electrodes or other aspects for applying an electrical field, wherein the electric field may destabilize the microdroplets such that they are merged together. Reagents may also, or instead, be added using picoinjection. In this approach, a target drop may be flowed past a channel containing the reagent(s) to be added, wherein the reagent(s) are at an elevated pressure. Due to the presence of the surfactants, however, in the absence of an electric field, the microdroplet will flow past without being injected, because surfactants coating the microdroplet may prevent the fluid(s) from entering. However, if an electric field is applied to the microdroplet as it passes the injector, fluid containing the reagent(s) will be injected into the microdroplet. The amount of reagent added to the microdroplet may be controlled by several different parameters, such as by adjusting the injection pressure and the velocity of the flowing drops, by switching the electric field on and off, and the like.

In some cases, a discrete entity includes a bead. In some cases, at least one dimension of the bead (e.g., diameter, is between about 0.5 μm and about 500 μm). In some cases, the bead is made of a polymeric material, such as polystyrene. In some cases, the bead is magnetic or contains a magnetic component. In some cases, the bead has a biomolecule attached to its surface, such as a barcoded oligonucleotide, an antibody, a protein, an antigen, DNA, RNA, streptavidin, or a combination thereof. In some cases, the bead is an immunoassay bead. In some cases, the bead is an RNA capture bead. As such, the present disclosure provides methods of selectively combining a biomolecule with another compound or cell, wherein the method includes selectively isolating the biomolecule from a composition using the bead, making a discrete entity that includes the bead and biomolecule, and selectively combining the discrete entity containing the bead and biomolecule with one or more other discrete entities that contain one or more other compounds or cells using the microfluidic device described herein. Methods of selectively isolating biomolecules using beads are known in the art, e.g. U.S. 2010/0009383, which is incorporated herein by reference for its disclosure of a method of separating a biomolecule or cell using beads.

In some embodiments, the methods, devices, and/or systems described herein can be used to detect nucleic acids, such as the neoantigen on the surface of the SD cells or the TCR from T-cells. In certain embodiments, reagents necessary for amplification are added to the droplets, either by combining them with the sample droplets prior to dispensing, or by dispensing additional droplets to the positions of the sample containing droplets, wherein the additional droplets include the necessary reagents and a detection component, where the detection component signals the amplification. The droplets are then incubated under conditions suitable for amplification and monitored to read the detection component. This provides, for each droplet, a rate of change of the detection component which can be used to detect and/or quantitate the nucleic acids in the droplets.

In some embodiments, the methods, devices, and/or systems described herein can be used to sequence nucleic acid derived from single cells. For example, individual cells (e.g., barcoded) can be encapsulated in the droplets and dispensed to the substrate as described herein. The barcoded cells can then be lysed and subjected to molecular biological processing to amplify and/or tag their nucleic acids with barcodes. The material from all the droplets can then be pooled for all cells and sequenced and the barcodes used to sort the sequences according to single droplets or cells. These methods can be used, for example, to sequence the genomes or transcriptomes of single cells in a massively parallel format.

As described above, in certain embodiments, nucleic acid sequence assay components that employ barcoding for labelling individual mRNA molecules, and/or for labeling for cell/well source (e.g., if wells pooled before sequencing analysis), and/or for labeling particular affixed entities (e.g., if droplet from two or more affixed entities are pooled prior to sequencing) are employed. Examples of such barcoding methodologies and reagents are found in Pat. Pub. US2007/0020640, Pat. Pub. 2012/0010091, U.S. Pat. Nos. 8,835,358, 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

In certain embodiments, the DropSeq method employing beads with primers attached to them are employed to sequence the nucleic acid from cells and associated barcodes. An example of such a method is described in Macosko et al., Cell, 161(5):1202-1214 (see, e.g., FIG. 1), which is herein incorporated by reference in its entirety. In certain embodiments employing DropSeq, barcoded template switch oligos are bound to beads and oligo dT is supplied in solution along with RT PCR reagents. Reverse transcription (RT) can, for example, be performed as described in Kim et al., Anal Chem. 2018 Jan. 16; 90(2):1273-1279, herein incorporated by reference. In other embodiments, barcoded oligo-dT beads are provided, the cells are lysed, mRNAs is captured on the beads, the emulsion is broken, and the drop is re-emulsified to capture mRNA beads with barcoded TSO beads where the TSO can be released by UV. Solution phase TSO can then be used for performing RT-PCR. Primers specific to the variable regions displayed on the surface of the SD cells can be employed to amplify such variable regions prior to sequencing.

In certain embodiments, unique oligo drops are provided to the fixed entities, and allow a link between imaging and genomics. For example, the unique oligos can contain two part 8 mer barcodes linked to polyA or TSO followed by 8-mer barcodes. In this regard, if one employs 96 barcoded oligos, selecting any three can generate 142,880 combinations. It is known what combination of three oligos are printed at each well position to identify that particular well (e.g., so a neoantigen that binds a TCR and activates the T-cell can be identified). These oligos will also be sequenced and so when one sees a particular 3-oligo combination in the sequencing readouts, one knows the fixed entity and the image for that fixed entity.

In certain embodiments, the barcode tagging and sequencing methods of WO2014201273 (“SCRB-seq” method, herein incorporated by reference) are employed. The necessary reagents for the SCRB-seq method (e.g., modified as necessary for small volumes) are added to the fixed entities, each containing a lysed cells. Briefly, the SCRB-seq method amplifies an initial mRNA sample from cells from a single fixed entity. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. The second primer is a template switching primer with a poly G 3′ end, and 5′ end that has iso-bases. After cDNA amplification, the tagged cDNA single fixed entity samples are pooled. Then full-length cDNA synthesis occurs with two different primers, and full-length cDNA is purified. Next, a NEXTERA sequencing library is prepared using an i7 primer (adds one of 12 i7 tags to identify particular multi-well plates) and P5NEXTPT5 to add P5 tag for NEXTERA sequencing (P7 tag added to the other end for NEXTERA). The library is purified on a gel, and then NEXTERA sequencing occurs. As a non-limting example, with twelve i7 plate tags, and 384 cell/well-specific barcodes, this allows total of 4,608 single cell transciptomes to be done at once. This method allows for quantification of mRNA transcripts in single fixed entity.

In other embodiments, the barcode tagging and sequencing methods employ concepts from the Multi-seq method. For example, cells are incubated with anchor and co-anchor lipid modified oligonucleotides (LMO) and encapsulated in droplets. Individual barcodes in droplets can hybridize to exposed regions of the LMOs and these barcodes can be used instead of Drop-seq beads. Anchor-coanchor LMOs remain bound to individual cells at 4° C. but can freely equilibrate between cells in a droplet at 37° C. Thus, a specific LMO-barcode combination in each droplet can be used to link two cells in that droplet that can be tracked after emulsion breaking. In one example, a unique LMO-barcode combination can be randomly assembled in every microfluidic droplet. Barcodes may also be deterministically pre-printed to a microwell array, and additionally provide linkage to imaging data recoded at specific microwell positions. In another embodiment, one cell in each combination may be LMO-barcoded before the combination in droplets. During incubation at 37° C., the LMO-barcodes will re-equilibrate to the initially non-barcoded cell and provide lasting information about co-encapsulation. If a unbarcoded T-cell is combined with an LMO-barcoded antigen presenting cell (APC), this process will allow the type of APC to be read out by sequencing only the T-cell.

In practicing the methods of the present disclosure, one or more sorting steps may be employed. A sorting step sorts a discrete entity into one of two or more locations (e.g. into one of two or more fluid channels). In some cases, the sorting is into one of two fluid channels. Discrete entities are sorted based on one or more properties of the discrete entity or a component within the discrete entity. In addition, such sorting may either be passive sorting or active sorting. Active sorting includes the detection of one or more properties of a discrete entity, or a component within the discrete entity, and sorting based on the detected property. Passive sorting involves sorting a discrete entity without the active detection of a property. Sorting approaches of interest include, by are not necessarily limited to, approaches that involve the use of one or more sorting channels and one or more sorting elements.

Sorting approaches which may be utilized in connection with the disclosed methods, systems and devices also include those described herein, and those described by Agresti, et al., PNAS vol. 107, no 9, 4004-4009. For active sorting, the device includes one or more sorting elements and one or more detectors, wherein each detector is configured to detect one or more properties of a discrete entity, or a component within the discrete entity, and each sorting element is configured to sort the discrete entity into one of two or more locations based on the detecting by the detection element. In some cases, a sorting element is positioned in proximity to the sorting channel, such as an electrode in proximity to the sorting channel. In some cases, a sorting element is positioned within the sorting channel, such as a partial height flow divider in a sorting channel. In some cases, the device includes a sorting element positioned within the sorting channel and one or more sorting elements positioned in proximity to the sorting channel. Exemplary structures and methods for active sorting discrete entities are described in Cole et al., PNAS, 2017, 114, 33, 8728-8733; Clark et al., Lab Chip, 2018, 5, 18, 710-713; and Sciambi et al., Lab on a Chip, 2015, 15, 47-51, the disclosures of which are incorporated herein by reference for sorting elements.

In some cases, the sorting element comprises an electrode configured to exert a dielectrophoretic force, an electrode configured to exert an electrophoretic force, an element configured to exert an acoustic force, a valve, or a combination thereof. In some cases, a sorting element comprises an electrode that is positioned in proximity to the sorting channel, e.g. an electrode configured to exert a dielectrophoretic force on the discrete entity or an electrophoretic force on the discrete entity. In some cases, the electrode is configured to exert an electrophoretic force on the discrete entity. The dielectrophoretic force on the discrete entity can be directed towards the electrode. In some cases, the sorting electrode is a liquid electrode, such as a microfluidic channel containing a conductive material, such as saltwater, liquid metal, molten solder, or a conductive ink to be annealed later. In some cases, the electrodes are micropatterned onto a planar surface and the microfluidic device is bonded to the surface. In some case, the electrodes are patterned on the substrate of the microfluidic device, e.g. a patterned indium tin oxide (ITO) glass slide. In some cases, the sorting element includes a selectively actuatable bipolar sorting electrode. In some cases, the sorting element includes two electrodes. In some cases, the sorting element includes a selectively actuatable bipolar droplet sorting electrode. In some cases, the electrode is a solid electrode prepared from any suitable conductive material may be utilized.

In some cases, the sorting element includes two sorting electrodes. In some cases, the two sorting electrodes have substantially different shapes, such as shown in FIG. 10. In some cases, the two sorting electrodes produce electric field lines with substantially different shapes. In some cases, the shapes are such that the pair of electrodes provide a constant electric field gradient. As such, a discrete entity can be subjected to the sorting force for a longer period of time and over a longer distance, thereby allowing a lower voltage to be used. In some cases, the electric field points radially inwards. In some cases, a portion of a first sorting electrode is positioned in the center of the arc of a concentric or essentially concentric sorting channel, and the second sorting electrode is positioned on a side of the sorting channel opposite the first sorting electrode, such as shown in FIG. 10. In some cases, the sorting channel defines a concentric or approximately concentric flow path, wherein a portion of a sorting electrode is located at the center of the concentric or approximately concentric flow path. In some cases, two sorting electrodes are positioned on the same side of the sorting channel. In such embodiments, the shortest distance between the two sorting electrodes is between about 20 μm and about 500 μm, such as between about 50 μm and about 200 μm, between about 75 μm and about 150 μm, between about 100 μm and about 150 μm, or between about 120 μm and about 140 μm. In some cases, the shortest distance between a sorting electrode and the interior of the sorting channel is between about 5 μm and about 100 μm, such as between about 10 μm and about 50 μm, between about 20 μm and about 40 μm, between about 25 μm and about 35 μm, or between about 28 μm and about 32 μm.

In some embodiments, the present disclosure provides microfluidic devices with an improved sorting architecture, which facilitates the high-speed sorting of discrete entities, e.g., microdroplets. This sorting architecture may be used in connection with other embodiments described herein or in any other suitable application where high-speed sorting of microdroplets is desired. Related methods and systems are also described. For example, in some embodiments, a microfluidic device may include a sorting channel; a first outlet channel in fluid communication with the sorting channel; a second outlet channel in fluid communication with the sorting channel; and a dividing wall separating the first outlet channel from the second outlet channel, wherein the dividing wall comprises a first proximal portion having a height which is less than the height of the inlet channel and a second distal portion having a height which is equal to or greater than the height of the inlet channel.

In some cases, the discrete entity is detected while the discrete entity is in the inlet channel via an optical property. In some cases, the optical property is fluorescence. Thus, in some cases, the detector includes an excitation light source and a fluorescence detector. In some cases, the excitation light includes visible light, ultraviolet light, or a combination thereof. In some cases, the detector is an optical scanner. In some cases, the detector includes optical fibers for directing excitation light onto the discrete entity, for directing fluorescent light to a fluorescence detector, or a combination thereof. In some cases, a suitable optical scanner utilizes a laser light.

A variety of different components can be included in the discrete entities to facilitate detection, including one or more fluorescent dyes. Such fluorescent dyes may be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va.

In some embodiments, the microfluidic devices herein include directing the discrete entity to a discrete entity merger region. Accordingly, a device as described herein can include a discrete entity merger region and a trapping element positioned in proximity to the discrete entity merger region. The trapping element can to trap a plurality of discrete entities in the discrete entity merger region for a time sufficient for the plurality of discrete entities to combine to form a combined discrete entity by exerting an electromagnetic force, exerting a mechanical force, applying heat, applying light, exerting an electrical force, providing a reagent, or a combination thereof sufficient. In some cases, the electromagnetic force is a dielectrophoretic force. In some cases, the electromagnetic force is an electrophoretic force. In some cases, the discrete entity merger region includes a feature selected from: a geometric change in a dimension of the first outlet channel, a flow obstacle, a flow divider, a laminating fluid inlet, a valve, or a combination thereof. In some cases, the geometric change is a change in the cross-sectional area of the first outlet channel (e.g., the discrete entity merger region has a larger cross-sectional area than the upstream region). In some cases, the geometric change is a change in one dimension of the first outlet channel (e.g., the discrete entity merger region is narrower than the downstream region). In some cases, the geometric change includes a recess in a channel wall. In some cases, the recess includes an area that is not colinear with the flow of fluid from the upstream region, such as shown as item 107 in FIG. 10. In some cases, where a valve is utilized, the valve is configured to switch between at least two states. In some cases, in the first state, the valve impedes the flow of a discrete entity past the discrete entity merger region while allowing flow of the carrier fluid past the discrete entity merger region. In some cases, in the second state, the valve is configured such that the combined discrete entity is not impeded from flowing past the discrete entity region. In some cases, the method includes putting the valve in a first state such that discrete entities can be trapped and combined into a combined discrete entity, and then putting the valve into a second state to release the discrete entity from the discrete entity merger region. In some cases, the valve is a membrane valve.

A laminating fluid inlet functions in a similar manner to certain embodiments of the spacer fluid inlet described above, such as a laminating fluid inlet is configured such that flowing fluid through the laminating fluid inlet will cause a discrete entity to move further away from a first side a channel and closer to a second side of a channel. Stated in another manner, the fluid flowing through the laminating fluid inlet contacts the fluid moving into the discrete entity merger region from an upstream region of the first outlet channel, thereby affecting the flow of fluid coming from the upstream region. In some cases, the fluid is oil, or a fluid which is otherwise immiscible with the fluid of the discrete entity.

FIG. 10 shows an embodiment wherein the discrete entity merger region includes recess 107, flow divider 113, and laminating fluid inlet 112. In FIG. 10, the laminating fluid provides a force pushing a discrete entity into recess 107 and towards trapping electrodes 109. In addition, flow divider 113 in FIG. 10 further affects the interaction of the laminating fluid and the fluid coming from the upstream region, thereby increasing the force pushing the discrete entity into recess 107. As such, a discrete entity merger region according to the present disclosure can include a laminating oil inlet and/or a flow divider, wherein such an element or elements are configured such that flowing oil through the laminating oil inlet channel will produce a force pushing a discrete entity in the discrete entity merger region towards a trapping electrode, a recess, or a combination thereof. In some embodiments, the device can include a flow divider without the laminating fluid inlet.

In some cases, the downstream region of the first outlet channel is configured to aid in the trapping of a discrete entity in the discrete entity merger region. In some cases, the downstream region has a larger cross-sectional area than the discrete entity merger region, which is an example of a geometric change in the first outlet channel. In some cases, the downstream region has a triangular or approximately triangular shape. In some cases, the downstream region has a triangular or approximately triangular shape and the discrete entity merger region is located at or near a vertex of the triangle. As an example, in the system of FIG. 11 has downstream region 208 and discrete entity merger region 207. In some cases, the longitudinal axis of the downstream region is parallel to the longitudinal axis of the discrete entity merger region, whereas in other cases such longitudinal axes are not parallel. In some cases, such axes are parallel but not colinear. In some cases, the axes are parallel and colinear. In some cases, the angle between such axes is greater than 0°, such as 5° or more, 10° or more, 15° or more, 30° or more, 45° or more, 60° or more, 75° or more, 90° or more, 135° or more, or 175° or more. In some cases, such an angle is between approximately 15° and approximately 135°. In some cases, such an angle is between approximately 60° and approximately 120°, such as shown in FIG. 3.

In some embodiments, the trapping element includes one or more electrodes, such as an electrode configured to exert a dielectrophoretic force on the discrete entity. In some cases, the electrode is configured to exert an electrophoretic force. The dielectrophoretic force on the discrete entity can be directed towards the electrode (i.e. an attractive force), away from the electrode (i.e. a repulsive force), or in any other direction. In some cases, the trapping electrode is a liquid electrode, such as a microfluidic channel containing a conductive material, e.g. saltwater, liquid metal, molten solder, or a conductive ink to be annealed later. In some case, the electrodes are patterned on the substrate of the microfluidic device, e.g. a patterned indium tin oxide (ITO) glass slide. In some cases, the trapping element includes a selectively actuatable bipolar trapping electrode. In some cases, the trapping element includes two electrodes. In some cases, is the trapping element includes a selectively actuatable bipolar droplet trapping electrode. In some cases, the electrode is a solid electrode prepared from any suitable conductive material may be utilized. In some cases, the trapping element includes three or more trapping electrodes, such as four or more, five or more, ten or more, or twenty or more. In such cases, the trapping electrodes can be configured to form two or more bipolar electrode pairs, such as three or more pairs, four or more pairs, five or more pairs, or ten or more pairs.

In some cases, the sorting element sorts discrete entities at a rate of at least 10 Hz, such as at least 100 Hz, at least 500 Hz, at least 1,000 Hz, at least 2,000 Hz, or at least 10,000 Hz. In some cases, only 50% or less of the discrete entities contain the contents desired for the second discrete entity, such as 25% or less, 10% or less, 5% or less, 1% or less, or 0.1% or less. In some cases, the discrete entity merger region and trapping element are configured to trap a first discrete entity for 0.1 ms or more, such as 1 ms or more, 5 ms or more, 10 ms or more, 25 ms or more, 50 ms or more, 100 ms or more, 500 ms or more, 1,000 ms or more, or 5,000 ms or more. In some cases, a first discrete entity is trapped in the discrete entity merger region for 0.1 ms or more before a second discrete entity enters the region, such as 1 ms or more, 10 ms or more, 100 ms or more, or 1,000 ms or more.

The present disclosure provides a method of selectively performing reactions by selectively combining two or more discrete entities, as described above, wherein the reaction occurs between one or more components from each discrete entity. Such components can be one or more cells, one or more products derived from a cell, one or more reagents, or a combination thereof. In some cases, a suitable method includes combination of one cell and one or more reagents. As an example, FIG. 12 shows the combination of four discrete entities, wherein three of the discrete entities each contain a different reagent and the fourth discrete entity contains a single cell. As such, FIG. 12 shows that a microfluidic device as described herein can be used to selectively combine different discrete entities, resulting in the formation of a combined discrete entity, e.g., that contains the three reagents and the cell. In some cases, the reagents can include cell lysing reagents, PCR reagents, reagents for analyzing the DNA or RNA within a cell, antibodies, or a combination thereof. In such cases, the method can further include collecting genomic data from contents of the discrete entities or combined discrete entities. In some cases, the one or more products derived from a cell include cell lysate, DNA, RNA, or a combination thereof. As such, the method can involve analyzing products from a cell, e.g. cell lysate, even though the cell per se is included in any of the discrete entities.

The present disclosure provides methods of selectively combining two or more discrete entities wherein each discrete entity contains one or more cell (e.g., a T-cell and a cell presenting a neoantigen). In some cases, the ratio of a first type of cell to a second type of cell is 1.1:1.0 or more, e.g. 2:1 or more, 5:1 or more, 10:1 or more, 25:1 or more. The number of cells can be 2:1, 2:1 or more, 5:1 or more, 10:1 or more, 25:1 or more. In other cases, three or more types of cells are combined in unequal ratios or numbers. The ratio or number of each pair of cells can be those numbers and ratios recited above.

In certain embodiments, gene signatures can be used to evaluate T-cells and NK cells (e.g., Chimeric Antigen Receptor-T cells' (CAR-T)) ability to secrete cytokines and identify T-cell specific, or NK-specific, signatures. Algorithms can be used to compute polyfunctionality and immune cell identity.

Polyfunctionality of a T-cell is defined as the T-cell's ability to secrete >2 cytokines/chemokines in response to specific T-cell perturbation. Polyfunctionality of T-cells in general and CAR-T cells in particular have been shown to be correlated to successful treatment outcomes depending on the type of cytokines they secrete. Cytokines have been categorized into effector, stimulatory, chemo-attractive, regulatory and inflammatory based on the impact they have on a patient. For example, T-cells that secrete high amounts of inflammatory cytokines can cause severe side effects whereas T-cells that secrete high amounts of effector cytokines are effective in killing its intended target.

Recent work from academia and industry have focused on evaluating secreted cytokines at a single-cell level using technologies such as single-cell barcoded chips (SCBC). SCBCs use single-cell ELISA methods to evaluate 30+ cytokines from 2000 single-cells and compute a polyfunctional score based on the different categories of cytokines. The polyfunctional scores are then used to identify responders and non-responders of CAR-T therapy.

Computing polyfunctional scores using protein measurements while robust also limits the subset of cytokines that one can interrogate to identify patient responders. An alternative approach is to use gene-expression signatures of single-cells. Single-cell RNA-seq methods have the potential to identify hundreds to thousands of differentially expressed genes. The subset of genes that can be used to evaluate polyfunctionality include genes that encode cytokines, transcription factors and other proteins such as annexin A1 that play a role in the regulation of T-cell activation.

Isoplexis uses single-cell cytokine measurements using ELISA, and therefore relies on secreted protein measurements. The methods described herein use gene expression measurements of single cells that have been sorted based on cytokine measurements. Such methods therefore takes into consideration both secreted and intracellular proteins to compute polyfunctionality.

In certain embodiments, the methods herein use microfluidics (e.g.,. as described elsewhere herein) to bring together patient T-cells (e.g., CAR-T cells), target cells and commercially available cytokine assay reagents. Such methods allow the ability to link functional analysis to single-cell genomics and VDJ sequencing.

In certain embodiments, cell-cell or cell protein release interactions are detected by proximity assays. Proximity assays rely on the interaction of two binding partners brought into close proximity by the target molecule (e.g., ATP, cytokines, granzyme B, and CD107a). Binding partners are typically nucleic acids or protein fragments. Binding partners can be bound directly or indirectly to detection reagents, such as antibodies or aptamers, or bind the target directly. When multiple detection reagents (with interaction partners bound to them) are both bound to the target, or when multiple interaction partners bind the target directly, the close proximity of the attached binding partners allows for covalent linkage (e.g. via ligation), hybridization, or general interaction. The interaction of the binding partners allows for detection via quantification of the bound partners, amplification of bound partners, or direct measurement using for example fluorescence (See, e.g., Xiao, Q., et al. (2018). “Multiplexed chemiluminescence imaging assay of protein biomarkers using DNA microarray with proximity binding-induced hybridization chain reaction amplification.” Anal Chim Acta 1032: 130-137; herein incorporated by reference in its entirety).

Fluorogenic and other activatable small-molecule detection strategies rely on direct modification of a substrate by the target of interest. Detection is performed directly on the modified substrate using for example fluorescence or absorbance. In certain embodiments, T-cell activation is based on granzyme B substrate cleavage detection. In some embodiments, a granzyme B substrate is included in a discrete entity or combined discrete entity. Examples of such granzyme B substrates includes, but are not limited to, Ac-IETD-AFC, Ac-IEPD-AMC, Ac-IETD-pNA, and Ac-IEPD-pNA.

### REFERENCES

1. Klein, et al., (2015). Droplet Barcoding for Single-Cell Transcriptomics Applied to Embryonic Stem Cells. Cell, 161(5), 1187-1201.
2. Lan, et al., (2016). Droplet barcoding for massively parallel single-molecule deep sequencing. Nature Communications, 7. https://doi.org/10.1038/ncomms11784
3. Buenrostro, et al., (2015). Single-cell chromatin accessibility reveals principles of regulatory variation. Nature, 523(7561), 486-490. https://doi.org/10.1038/nature14590
4. Rossi, et al., (2018). Preinfusion polyfunctional anti-CD19 chimeric antigen receptor T cells are associated with clinical outcomes in NHL. Blood, 132(8), 804-814.
5. Method of the Year 2019: Single-cell multimodal omics. (2020, January 1). Nature Methods, Vol. 17, p. 1. https://doi.org/10.1038/s41592-019-0703-5
6. Cohen, et al., (2018). Lung Single-Cell Signaling Interaction Map Reveals Basophil Role in Macrophage Imprinting In Brief. Cell, 175, 1031-1044.e18.
7. Halpern, et al., (2018). Paired-cell sequencing enables spatial gene expression mapping of liver endothelial cells. Nature Biotechnology, (September). https://doi.org/10.1038/nbt.4231
8. Moncada, et al., (2020). Integrating microarray-based spatial transcriptomics and single-cell RNA-seq reveals tissue architecture in pancreatic ductal adenocarcinomas. Nature Biotechnology. https://doi.org/10.
9. Gracz, et al., (2015). A high-throughput platform for stem cell niche co-cultures and downstream gene expression analysis. Nat Cell Biol, 17(3), 340-349.
10. Mocciaro, et al., (2018). Light-activated cell identification and sorting (LACIS) for selection of edited clones on a nanofluidic device. Communications Biology, 1(1).
11. McGinnis, et al., (2019). MULTI-seq: sample multiplexing for single-cell RNA sequencing using lipid-tagged indices. Nature Methods, 16(7), 619-626.
12. Stoeckius, et al., (2018). Cell Hashing with barcoded antibodies enables multiplexing and doublet detection for single cell genomics. Genome Biology, 19(1), 224.
13. Kula, et al., (2019). T-Scan: A Genome-wide Method for the Systematic Discovery of T Cell Epitopes. Cell, 178(4), 1016-1028.e13.
14. Li, et al., (2019). T cell antigen discovery via trogocytosis. Nature Methods, 16(2), 183-190.
15. Stoeckius, et al., (2017). Simultaneous epitope and transcriptome measurement in single cells. Nature Methods, 14(9), 865-868.
16. WO2020010366
17. WO2019023269
18. US Pat. Pub. 201810251825

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising:
a) a carrier fluid,
b) a discrete entity in said carrier fluid,
c) a cell and a secreted protein from said cell in said discrete entity,
d) a capture bead configured to bind to said secreted protein in said discrete entity, and
e) a plurality of antibody-linked oligonucleotides (ALOs) in said discrete entity, wherein each ALO:
i) comprises a nucleic acid sequence comprising the same barcode region, and
ii) an antibody, or a binding region of said antibody, linked to said nucleic acid sequence, wherein said antibody, and said binding region, specifically bind to said secreted protein.

2. The composition of claim 1, wherein said secreted protein is selected from the group consisting of: a cytokine, a hormone, an enzyme, a toxin, and an antimicrobial peptide.

3. The composition of claim 1, wherein said nucleic acid sequence further comprises: a first amplification handle and a first anchor.

4. The composition of claim 3, wherein said first anchor comprises a poly-A sequence.

5. The composition of claim 1, wherein said first and second cells are present in said first discrete entity, and said plurality of ALOs are present in said second discrete entity.

6. The composition of claim 1, wherein said discrete entity is a droplet.

7. The composition of claim 6, wherein said droplet comprises an aqueous fluid which is immiscible in said carrier fluid.

8. A method comprising:
a) merging first, second, and third discrete entities in a carrier fluid in a microfluidics device such that a combined discrete entity is formed,
wherein said first discrete entity contains a cell that secretes a protein of interest,
wherein said second discrete entity contain a bead that binds to said protein of interest, and
wherein said third discrete entity contains a plurality of antibody-linked oligonucleotides (ALOs), wherein each ALO: i) comprises a nucleic acid sequence comprising the same barcode region, and ii) an antibody, or a binding region of said antibody, linked to said nucleic acid sequence, wherein said antibody, or binding region of said antibody, binds said protein of interest; and
b) incubating said combined discrete entity such that: i) said cell secretes said protein of interest, ii) said bead binds said protein of interest, and iii) an said binds to said protein of interest.

9. The method of claim 8, wherein said cell and/or said capture bead are labeled with a moiety linked to a barcoded nucleic acid sequence, wherein said moiety is selected from: a lipid, cholesterol, a surface molecule, an antibody, or a binding region of said antibody.

10. The method of claim 8, wherein said secreted protein is selected from the group consisting of: a cytokine, a hormone, an enzyme, a toxin, and an antimicrobial peptide.

11. The method of claim 8, wherein said cell is a T-Cell or an antigen presenting cell (APC).

12. The method of claim 8, further comprising: c) processing said combined discrete entity such that: i) first sequencing information is obtained from said cell, and ii) separately, second sequence information is obtained from said barcode region.

13. The method of claim 12, further comprising: d) identifying said second sequence, and therefore said secreted protein, as originating from said cell in said combined discrete entity.

14. The method of claim 8, wherein said cell and said capture bead are labeled with a moiety linked to a barcoded nucleic acid sequence, wherein said moiety is selected from: a lipid, cholesterol, a surface molecule, an antibody, or a binding region of said antibody.

15. A system comprising:
a) a carrier fluid,
b) first, second, and third discrete entities, or a combined discrete entity, in said carrier fluid, wherein said combined discrete entity is composed of said first discrete entity and either said second or third discrete entity,
c) first and second cells in said first discrete entity or in said combined discrete entity,
d) a plurality of first antibody-linked oligonucleotides (1st-ALOs) in said second discrete entity, or in said combined discrete entity, wherein each 1st-ALO:
i) comprise a first nucleic acid sequence comprising a first barcode region, and
ii) a first antibody, or a binding region of said first antibody, linked to said first nucleic acid sequence, wherein said first antibody and said binding region specifically bind to the surface of said first and second cells; and
e) a plurality of second antibody-linked oligonucleotides (2nd-ALOs) in said third discrete entity, or in said combined discrete entity, wherein each 2nd-ALO:
i) comprise a second nucleic acid sequence comprising a second barcode region different from said first barcode region, and
ii) a second antibody, or a binding region of said second antibody, linked to said second nucleic acid sequence, wherein said second antibody and said binding region specifically bind to the surface of said first and second cells.

16. The system of claim 15, wherein said first cell is able to be activated by said second cell to release a detectable signal.

17. The system of claim 15, wherein said first discrete entity further comprises detection reagents to detect said detectable signal.

18. The system of claim 15, wherein said first and second cells in said combined discrete entity are labeled with said 1st-ALOs.

19. The system of claim 15, wherein said first and second cells in said combined discrete entity are labeled with said 2nd-ALOs.

20. The system of claim 15, wherein one of said first and second cells is a T-cell and the other is an antigen presenting cell.
